(19) United States
(12) Patent Application Publication
(10) Pub. No.: US 2012/0116050 A1
(43) Pub. Date: May 10, 2012

(54) PYRENE-BASED POLYMERS FOR ORGANIC LIGHT EMITTING DIODES (OLEDS)

(75) Inventors: Klaus Muellen, Koeln (DE); Teresa Figueira-Duarte, Mainz (DE); Pablo Gabriel Del Rosso, Bahia Blanca (AR); Emil J.W. List, Graz (AT); Roman Trattnig, Klagenfurt (AT)

(73) Assignees: Technische Universitaet Graz, Graz (AT); Max-Planck-gesellschaft zur Foerderung der Wissenschaften e.V., Muenchen (DE)

(21) Appl. No.: 13/377,947

(22) PCT Filed: Apr. 20, 2010
(86) PCT No.: PCT/EP2010/002414

§ 371 Date: Jan. 26, 2012

(30) Foreign Application Priority Data
Jun. 15, 2009 (EP) 09007843.7

(51) Int. Cl. C08G 61/10; C07C 1/20

(52) U.S. Cl. 528/396; 585/469

(57) ABSTRACT

The present invention relates to novel pyrene-based polymers, methods of preparing the same and uses thereof, in particular for electroluminescent devices. The novel polymers of the invention have the following general formula (I): wherein R¹, R², R³, R⁴, R⁵, R⁶, R⁷ and R⁸ are independently of each other hydrogen, halogen, in particular F, SiR¹⁰⁰R¹⁰¹R¹⁰², or an organic substituent, or R⁶ and R⁷, R³ and R⁴, and/or any of the substituents R¹, R², R³, R⁴, R⁵, R⁶, R⁷ and/or R⁸, which are adjacent to each other, together form an aromatic, or heteroaromatic ring, or ring system, which can optionally be substituted, n₁ and n₂ are 0, 1, or 2, R¹⁰⁰, R¹⁰¹ and R¹⁰² are independently of each other C₁-C₁₈ alkyl, substituted or unsubstituted C₆-C₁₈ aryl, and Ar¹ and Ar² are each independently of each other a substituted or unsubstituted arylene or heteroarylene group.

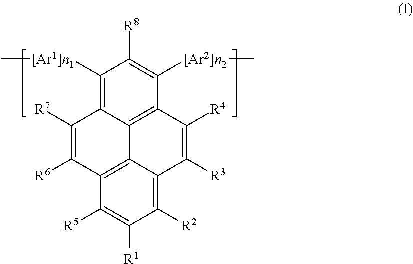

Pyrene is one of the most important and thoroughly investigated organic chromophores. Among the attractive features of pyrene is its exceptionally long fluorescence lifetime, the sensitivity of its excitation spectra to microenvironment changes, and its high propensity for forming excimers. This excimer formation has been utilized over the last 50 years in the investigation of water-soluble polymers, making pyrene, by far, the most frequently applied dye in fluorescence labeled polymers.

Despite its chemical stability and high quantum yield, the formation of excimers has also prohibited its use as an emissive material in organic light-emitting devices (OLED)s. Since the report of the first double-layer thin-film OLED by the Kodak Company in 1987, OLEDs have attracted enormous attention in the scientific community thanks to their high technological potential for the next generation of full-color-flat-panel displays and lighting applications. Whether polymers or small molecules, to date only red and green emitters have shown sufficient efficiencies and lifetimes to be of commercial value.

In recent years, there has been an increasing interest in the use of pyrene units in the synthesis of emissive and charge transport materials for OLEDs, including oligothiophenes with pyrenyl side groups or end-groups, bipyrenylbenzene molecules, as much as pyrene-carbazole and pyrene-fluorene systems. However, the pyrene derivatives which have been reported so far as efficient blue emitters for OLED applications present some degree of aggregation in the solid state.

U.S. Pat. No. 6,852,429 B1 claims a non-polymeric pyrene-based compound having several bulky substituents and its use in an organic light emitting device. The presence of said bulky substituent groups is said to reduce intermolecular aggregation as compared to 1,3,5-tripyrene benzene (3TPB). US 2006/0113528 A1 discloses an organic light-emitting device wherein a light emitting region in at least one layer of said device comprises i.a. a complex organic compound comprising up to 3 pyrene units directly or via bridging groups linked to a (further substituted) anthracene unit. US 2008/0166595 A1 discloses electroluminescent 4,9-di-substituted pyrenes and electronic devices in which the active layer includes such a pyrene composition.

The most successful effort in the prevention of aggregation in small molecules was achieved with tetra-substituted highly sterically congested pyrenes (Sotoyama et al., Tetra-substituted pyrenes: new class of blue emitter for organic light-emitting diodes. SID Digest 45, 1294-1297 (2003)), being the most well-known case the 1,3,6,8-tetraphenylpyrene with application in OLEDs, organic field effect transistors (OFET)s as well in organic light emitting field effect transistors (OLEFET)s. Additional tetra-substituted systems including different phenyl derivatives or pyridyl units at the 1,3,6,8 positions have been reported as well. Recently, the present inventors reported the supression of aggregation in a strongly twisted multichromophoric dendrimer made up exclusively from pyrene units at the 1,3,6,8-positions of the pyrene ring, which revealed a very high fluorescence quantum yield relative to unsubstituted pyrene (Müllen et al., Polypyrene Dendrimers. Angew. Chem. Int. Ed. 47, 10175-10178 (2008)). Furthermore, 1,1′-bipyrenyl and linear 1,6-disubstituted oligopyrenes were investigated.

In comparison to small molecules, conjugated organic polymers have the advantage to access larger display sizes and lighting devices at much lower manufacturing costs via solution-based deposition techniques. Only a small number of investigations concerning the attachment of pyrene to the polymeric chain or the use of pyrene along the polymeric backbone were reported as new materials for molecular electronics.

EP0964045 describes polymeric fluorescent substances of the formula —Ar₁—CR₁═CR₂—, wherein Ar₁ may be a pyrene unit substituted at the 1,6- or 1,8-position of the pyrene ring. In a recent International patent application by Schäfer, Müllen et al. (WO 2008/012250) fluorescent polymers comprising 2,7-linked pyrene units are disclosed.

In particular with respect to pyrene homopolymers, a few publications described the preparation of polypyrene via the electrochemical polymerization of pyrene (Bargon et al., Electrochemical synthesis of electrically conducting polymers from aromatic compounds. IBM J. Res. Develop. 27, 330-341 (1983); Hino et al., Ultraviolet photoelectron spectra of electropolymerized polymers: polyazulene, polypyrene and polycarbazole. Synt. Met. 64, 259-264 (1994), but the products exhibited only extremely low molecular weights. Polypyrenes formed via electrochemical polymerization of pyrenes by 1-1′ coupling were described to give as insoluble and unprocessable film or alternatively to give soluble materials with few repeat units. Thereby, the low degree of polymerization is presumed to be a consequence of the low solubility caused by the strong self-assembly of pyrene segments.

Summarizing, most of the oligomers and polymers of the prior art which comprise or consist of pyrene repeating units show a certain degree of aggregation and due to this aggregation phenomenon the emission quantum yield is relatively low and red-shifted.

Thus, an object of the present invention is to provide novel pyrene-based homopolymers and copolymers having improved properties, such as lack of aggregation, high electro-luminescence, high emission quantum yield, color purity and solubility which are particular suitable for organic light emitting diodes (OLEDs) and related electronic devices, as well as, a method for preparing the same.

Said object is achieved according to the present invention in particular by the compounds according to claims 1-13, the methods according to claims 14-16, and the uses and devices according to claims 17-21.

The new compounds are characterized by one or more 1,3-substituted (and linked) pyrene unit(s) as shown in formula I below which may be linked with each other or additional arylene or heteroarylene units and/or with other comonomeric units. The substitution at the 1,3-position of the pyrene ring surprisingly results in a highly twisted structure, which avoids aggregation and provides high emission quantum yields.

The polymers according to the invention exhibit high chemical stability, excellent control over electrical, optical and morphological properties in thin films resulting in high and stable electroluminescence. Specifically, the suppression of unwanted aggregation in thin films leads to highly efficient electroluminescence and blue-emission in the case of the homopolymer. In addition, high charge carrier mobilities and high temperature stability of the emission color can be observed, if the polymers according to the invention are used in polymer light emitting diodes (PLEDs). Organic light emitting diodes (OLEDs), comprising the polymers of the present invention, can show significant advantages in color purity, device efficiency and/or operational lifetime. In addition, the polymers can have good solubility characteristics in most organic solvents and high glass transition temperatures, which facilitates their fabrication into coatings and thin films, that are thermally and mechanically stable and relatively free of defects. If the polymers contain end groups which are capable of being crosslinked, the crosslinking of such groups after the films or coating is formed increases the solvent resistance thereof, which is beneficial in applications wherein one or more solvent-based layers of material are deposited thereon.

The fluorescent polymer of the invention according to claim 1 comprises one or more repeating units shown in the following general formula (I)

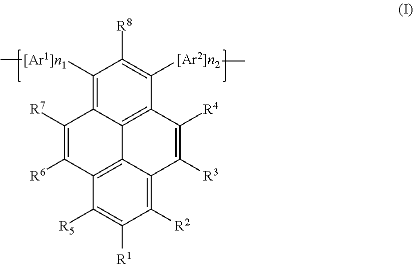

wherein R¹, R², R³, R⁴, R⁵, R⁶, R⁷ and R⁸ are independently of each other hydrogen, halogen, SiR¹⁰⁰R¹⁰¹R¹⁰², or an organic substituent, or
R⁶ and R⁷, R³ and R⁴, and/or any of the substituents R¹, R², R³, R⁴, R⁵, R⁶, R⁷ and/or R⁸, which are adjacent to each other, together form an aromatic, or heteroaromatic ring, or ring system, which can optionally be substituted, n₁ and n₂ are 0, 1, or 2,
R¹⁰⁰, R¹⁰¹ and R¹⁰² are independently of each other C₁-C₁₈ alkyl, substituted or unsubstituted C₆-C₁₈ aryl, and Ar¹ and Ar² are each independently of each other a substituted or unsubstituted arylene, or heteroarylene group.

In a more specific embodiment of the fluorescent polymer according to the invention R¹, R², R³, R⁴, R⁵, R⁶, R⁷, and R⁸ are independently of each other H, halogen, in particular F, C₁-C₁₈ alkyl, C₁-C₁₈ alkyl which is substituted by E and/or interrupted by D, C₁-C₁₈ perfluoroalkyl, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, CN, or —CO—R²⁸,

### D is —CO—; —COO—; —S—; —SO—; —SO2—; —O—; —NR²⁵—; —SiR³⁰R³¹—; —POR³²—; —CR²³═CR²⁴—; or C—≡ C—; and

E is —OR²⁹; —SR²⁹; —NR²⁵R²⁶; —COR²⁸; —COOR²⁷; —CONR²⁵R²⁶; —CN; or halogen, especially F;
G is E, C₁-C₁₈ alkyl, C₁-C₁₈ alkyl which is interrupted by D, C₁-C₁₈ perfluoroalkyl, C₁-C₁₈ alkoxy, or C₁-C₁₈ alkoxy which is substituted by E and/or interrupted by D,
R²³, R²⁴, R²⁵ and R²⁶ are independently of each other H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—;
R²⁷ is H; C₆-C₁₈ aryl; C₆-C₁₈ aryl which is substituted by C₁-C₁₈ alkyl, or C₁-C₁₈ alkoxy; especially C₁-C₁₈ alkyl; or C₁-C₁₈ alkyl which is interrupted by —O—,
R²⁸ is H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—,
R²⁹ is H; C₆-C₁₈ aryl; C₆-C₁₈ aryl, which is substituted by C₁-C₁₈ alkyl, or C₁-C₁₈ alkoxy; C₁-C₁₈ alkyl; or C₁-C₁₈ alkyl which is interrupted by —O—,
R³⁰ and R³¹ are independently of each other C₁-C₁₈ alkyl, C₆-C₁₈ aryl, or C₆-C₁₈ aryl, which is substituted by C₁-C₁₈ alkyl, and
R³² is C₁-C₁₈ alkyl, C₆-C₁₈ aryl, or C₆-C₁₈ aryl, which is substituted by C₁-C₁₈ alkyl.

Specifically, R¹ is C₁-C₁₈ alkyl, C₆-C₂₄ aryl or C₆-C₂₄ aryl which is substituted by G as defined above. Preferably R¹ is a C₁-C₁₂ alkyl group, more preferred a branched lower alkyl group of C₁-C₈, such as a tert-alkyl group, in particular a tert-butyl group.

R³ and R⁴ as well as R⁶ and R⁷ can be different from each other, but are preferably the same. Most preferred R³, R⁴, R⁶ and R⁷ have the same meaning.

R³, R⁴, R⁶ and R⁷ and R², R⁵, R⁸ are preferably selected from H, C₁-C₁₈ alkyl, C₁-C₁₈ alkyl which is substituted by E and/or interrupted by D; C₁-C₁₈ alkoxy, C₁-C₁₈ alkoxy, which is substituted by E and/or interrupted by D; C₁-C₁₈ perfluoroalkyl or an optionally substituted C₆-C₂₄ aryl, or C₂-C₂₀ heteroaryl group.

In a specific embodiment of the present invention at least one, very especially at least two of R³, R⁴, R⁶ and R⁷ are different from H. More specifically, all of the substituents R³, R⁴, R⁶ and R⁷ are different from H. In another specific embodiment of the present invention at least one, preferably two of the substituents R³, R⁴, R⁶ and R⁷ are an optionally substituted C₁-C₁₈ alkoxy group. More specifically, all of the substituents R³, R⁴, R⁶ and R⁷ are an optionally substituted C₁-C₁₈ alkoxy group.

Preferably, the polymer of the present invention comprises repeating unit(s) of formula I, wherein R³, R⁴, R⁶ and R⁷ are independently of each other H, F, C₁-C₁₈ alkyl, C₁-C₁₈ alkyl which is substituted by E and/or interrupted by D, C₁-C₁₈ perfluoroalkyl, C₆-C₂₄ aryl, C₆-C₂₄ aryl which is substituted by G, C₂-C₂₀ heteroaryl, C₂-C₂₀ heteroaryl which is substituted by G; each group R₅ and R₆ is independently of each other in each occurrence H, halogen, especially F, C₁-C₁₈ alkyl, C₁-C₁₈ alkyl which is substituted by E and/or interrupted by D, C₁-C₁₈ perfluoroalkyl, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, CN, or —CO—R²⁸,

### D is —CO—; —COO—; —S—; —SO—; —SO2—; —O—; —NR²⁵—; —SiR³⁰R³¹—; —POR³²—; —CR²³═CR²⁴—; or —C≡ C—; and

E is —OR²⁹; —SR²⁹; —NR²⁵R²⁶; —COR²⁸; —COOR²⁷; —CONR²⁶R²⁶; —CN; or halogen, especially F;
G is E, C₁-C₁₈ alkyl, C₁-C₁₈ alkyl which is interrupted by D, C₁-C₁₈ perfluoroalkyl, C₁-C₁₈ alkoxy, or C₁-C₁₈ alkoxy which is substituted by E and/or interrupted by D,
R²³, R²⁴, R²⁵ and R²⁶ are independently of each other H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—;
R²⁷ is H; C₆-C₁₈ aryl; C₆-C₁₈ aryl which is substituted by C₁-C₁₈ alkyl, or C₁-C₁₈ alkoxy; especially C₁-C₁₈ alkyl; or C₁-C₁₈ alkyl which is interrupted by —O—,
R²⁸ is H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—,
R²⁹ is H; C₆-C₁₈ aryl; C₆-C₁₈ aryl, which is substituted by C₁-C₁₈ alkyl, or C₁-C₁₈ alkoxy; C₁-C₁₈ alkyl; or C₁-C₁₈ alkyl which is interrupted by —O—,
R³⁰ and R³¹ are independently of each other C₁-C₁₈ alkyl, C₆-C₁₈ aryl, or C₆-C₁₈ aryl, which is substituted by C₁-C₁₈ alkyl, and R³² is C₁-C₁₈ alkyl, C₆-C₁₈ aryl, or C₆-C₁₈ aryl, which is substituted by C₁-C₁₈ alkyl.

Preferably, R¹, R², R³, R⁴, R⁵, R⁶, R⁷ and R⁸ are independently of each other H, C₁-C₁₈ alkyl, such as methyl, ethyl, n-propyl, iso-propyl, n-butyl, isobutyl, sec-butyl, t-butyl, 2-methyl-butyl, n-pentyl, isopentyl, n-hexyl, 2-ethylhexyl, or n-heptyl; C₁-C₁₈ alkyl which is substituted by E and/or interrupted by D, such as —CH₂OCH₃, —CH₂OCH₂CH₃, —CH₂OCH₂CH₂OCH₃, or —CH₂OCH₂CH₂OCH₂CH₃; C₁-C₁₈ alkoxy, such as methoxy, ethoxy, n-propoxy, iso-propoxy, n-butoxy, isobutoxy, sec-butoxy, t-butoxy, 2-methylbutoxy, n-pentyloxy, isopentyloxy, n-hexyloxy, 2-ethylhexyloxy, or n-heptyloxy; C₆-C₁₄ aryl, such as phenyl, naphthyl, or biphenylyl, C₅-C₁₂ cycloalkyl, such as cyclohexyl, C₆-C₁₄ aryl which is substituted by G, such as —C₆H₄OCH₃, —C₆H₄OCH₂CH₃, —C₆H₃(OCH₃) ², or —C₆H₃(OCH₂CH₃)₂, —C₆H₄CH₃, —C₆H₃(CH₃)₂, —C₆H₂(CH₃)₃, —C₆H₄OtBu, or —C₆H₄tBu.

D is preferably —CO—, —COO—, —S—, —SO—, —SO2—, —O—, —NR²⁵—, wherein R²⁵ is C₁-C₁₂ alkyl, such as methyl, ethyl, n-propyl, iso-propyl, n-butyl, isobutyl, tert-butyl or sec-butyl, or C₆-C₁₄ aryl, such as phenyl, naphthyl, or biphenylyl.

E is preferably —OR²⁹; —SR²⁹; —NR²⁵R²⁵; —COR²⁸; —COOR²⁷; —CONR²⁵R²⁵; or —CN; wherein R²⁵, R²⁷, R²⁸ and R²⁹ are independently of each other C₁-C₁₂ alkyl, such as methyl, ethyl, n-propyl, iso-propyl, n-butyl, isobutyl, sec-butyl, tert-butyl, hexyl, octyl, or 2-ethylhexyl, or C₆-C₁₄ aryl, such as phenyl, naphthyl, or biphenylyl.

G has the same preferences as E, or is C₁-C₁₈ alkyl, especially C₁-C₁₂ alkyl, such as methyl, ethyl, n-propyl, iso-propyl, n-butyl, isobutyl, sec-butyl, tert-butyl, hexyl, octyl, 1-(2-hexyl)decane, or 2-ethylhexyl.

Specifically, Ar¹ and Ar² are selected from a substituted or unsubstituted benzene, a substituted or unsubstituted naphthalene, a substituted or unsubstituted anthracene, a substituted or unsubstituted diphenylanthracene, a substituted or unsubstituted phenanthrene, a substituted or unsubstituted triphenylene, a substituted or unsubstituted acenaphthene, a substituted or unsubstituted biphenyl, a substituted or unsubstituted fluorene, a substituted or unsubstituted carbazolyl, a substituted or unsubstituted thiophene, substituted or unsubstituted multi-fused thiophenes, a substituted or unsubstituted triazole, a substituted or unsubstituted thiadiazole, a substituted or unsubstituted pyrene, a substituted or unsubstituted triphenylamine, or another chromophore, for example a perylenediimide or perylenemonoimide or higher rylene homologues thereof.

More specifically, Ar¹ and Ar² may be independently selected from the following formulae

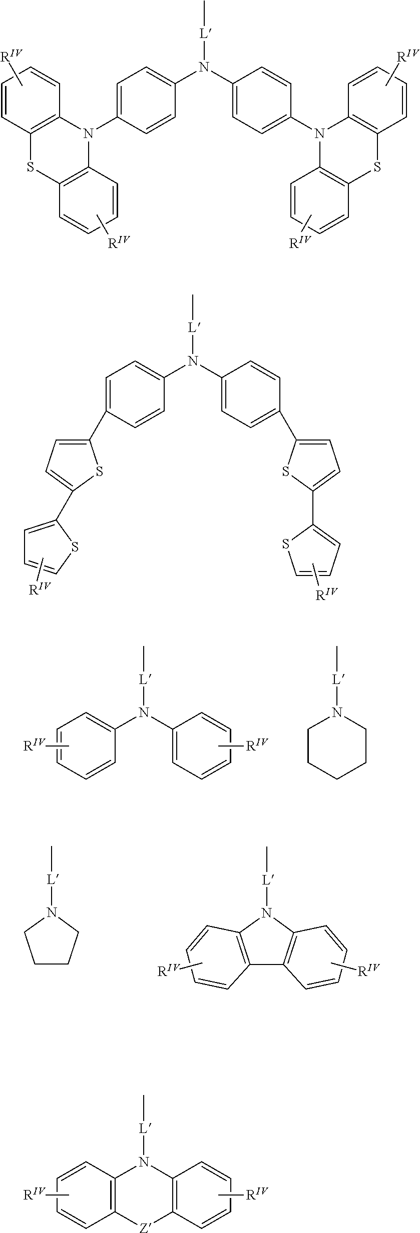

where the variables are each defined as follows:
L″ is a chemical bond or 1,4-phenylene;
Z″ is —O—, —S—, NR^{8′} or —CH₂—, where R^{8′} is C₁-C₁₈-alkyl;
R^{IV} is C₄-C₁₈-alkyl, C₁-C₁₈-alkoxy, (hetero)aryl, or —NR⁵R⁶ with R⁵ and R⁶ independently are as defined above.
or from the following formulae:

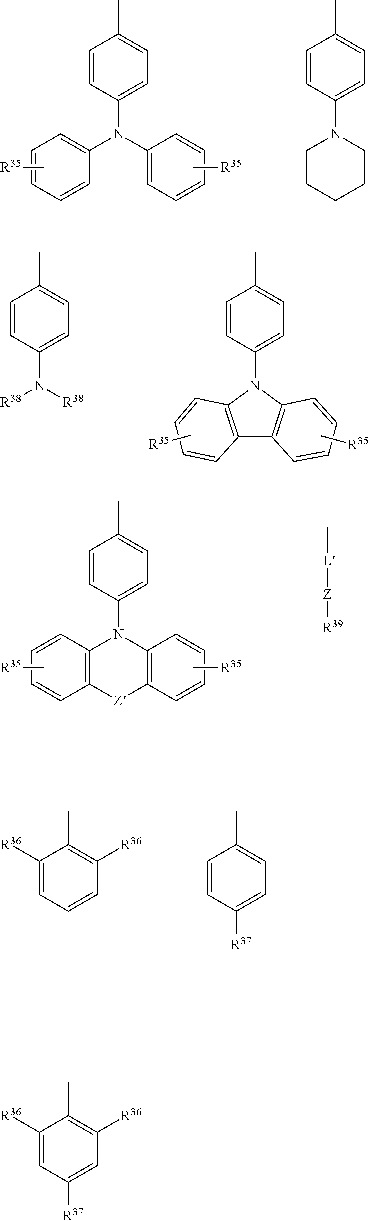

where the variables are each defined as follows:
R³⁵ is C₄-C₁₈-alkyl or C₁-C₁₈-alkoxy;
R³⁶ is C₃-C₈-alkyl, preferably with a secondary carbon atom in the 1-position;
R³⁷ is C₄-C₁₈-alkyl, preferably with a tertiary carbon atom in the 1-position or NR⁹R¹⁰;
R³⁸ is C₁-C₁₈-alkyl;
R³⁹ is phenyl when L′ is a chemical bond;
C₄-C₁₈-alkyl when L′ is 1,4-phenylene;
L′ is a chemical bond, 1,4-phenylene or 2,5-thienylene;
Z′ is —O—; —S—, —NR^{8′}— or —CH₂—, where R^{8′}, R^{9′} and R^{10′} are C₁-C₁₈-alkyl;

### Z is —O— or —S—;

or from the following formulae:

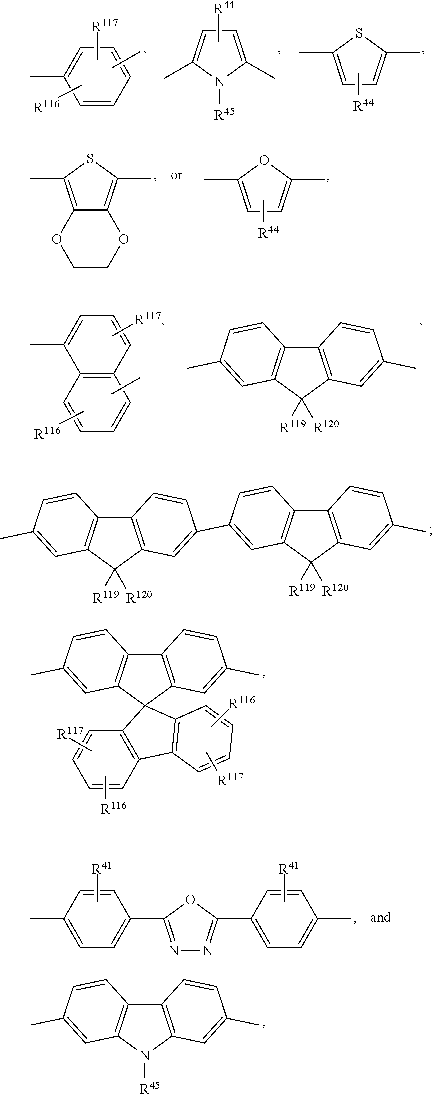

wherein
R⁴⁴ and R⁴¹ are hydrogen, C₁-C₁₈ alkyl, or C₁-C₁₈ alkoxy, and R⁴⁵ is H, C₁-C₁₈ alkyl, or C₁-C₁₈ alkyl which is substituted by E and/or interrupted by D, especially C₁-C₁₈ alkyl which is interrupted by —O—,
R¹¹⁶ and R¹¹⁷ are independently of each other H, halogen, —CN, C₁-C₁₈ alkyl, C₁-C₁₈ alkyl which is substituted by E and/or interrupted by D, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, —C(═O)—R¹²⁷, —C(═O)OR¹²⁷, or —C(═O)NR¹²⁷R¹²⁶,
R¹¹⁹ and R¹²⁰ are independently of each other H, C₁-C₁₈ alkyl, C₁-C₁₈ alkyl which is substituted by E and/or interrupted by D, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, or C7-C25 aralkyl, or R¹¹⁹ and R¹²⁰ together form a group of formula ═CR¹²¹R¹²², wherein R¹²¹ and R¹²² are independently of each other H, C₁-C₁₈ alkyl, C₁-C₁₈ alkyl which is substituted by E and/or interrupted by D, C₆-C₂₄ aryl, C₆-C₂₄ aryl which is substituted by G, or C₂-C₂₀ heteroaryl, or C₂-C₂₀ heteroaryl which is substituted by G,
or R¹¹⁹ and R¹²⁰ together form a five or six membered ring, which optionally can be substituted by C₁-C₁₈ alkyl, C₁-C₁₈ alkyl which is substituted by E and/or interrupted by D, C₆-C₂₄aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, or —C(═O)—R¹²⁷, and
R¹²⁶ and R¹²⁷ are independently of each other H; C₆-C₁₈ aryl; C₆-C₁₈ aryl which is substituted by C₁-C₁₈ alkyl, or C₁-C₁₈ alkoxy;
C₁-C₁₈ alkyl; or C₁-C₁₈ alkyl which is interrupted by —O—,
wherein G, D and E are as defined above.

The present invention also provides monomers for the preparation of the above polymers of the invention and such monomers form a further embodiment of the present invention. The monomers are represented by the following formula

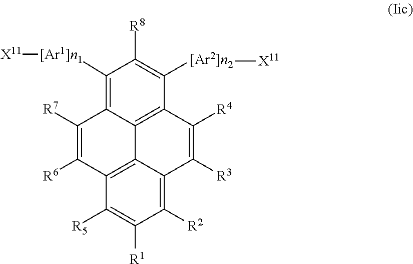

wherein Ar¹, Ar², n₁, n₂, R¹, R², R³, R⁴, R⁵, R⁶, R⁷, R⁸ are as defined above. X¹¹ is independently in each occurrence a halogen atom, especially I, Cl, or Br; —ZnX¹², —SnR²⁰⁷R²⁰⁸R²⁰⁹, wherein R²⁰⁷, R²⁰⁸ and R²⁰⁹ are identical or different and are H or C₁-C₆ alkyl, wherein two radicals optionally form a common ring and these radicals are optionally branched or unbranched and X¹² is a halogen atom, very especially I, or Br; or —OS(O)₂CF₃, —OS(O)₂-aryl, especially

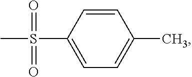

### —OS(O)₂CH₃, —B(OH)₂, —B(OY¹¹)₂,

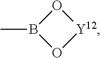

—BF₄Na, or —BF₄K, wherein Y¹¹ is independently in each occurrence a C₁-C₁₀ alkyl group and Y¹² is independently in each occurrence a C₂-C₁₀ alkylene group, such as —CY¹³Y¹⁴—CY¹⁵Y¹⁶—, or —CY¹⁷Y¹⁸—CY¹⁹Y²⁰—CY²¹Y²²—, wherein Y¹³, Y¹⁴, Y¹⁵, Y¹⁶, Y¹⁷, Y¹⁸, Y¹⁹, Y²⁰, Y²¹ and Y²² are independently of each other hydrogen, or a C₁-C₁₀ alkyl group, especially —C(CH₃)₂C(CH₃)₂—, or —C (CH₃)₂CH₂C(CH₃)₂—.

The attachment of one or more alkyl chains, including branched alkyl chains, to a pyrene ring at a specific position(s) can be effected by various means, for example the inventors developed a new method to attach alkyl chains to the 2- and 2,7-positions of the pyrene ring, starting from the pyrene-2-boronate (IIia) and pyrene-2,7-bis(boronate) (IIia′) reported by Conventry et al. (Chem. Commun. 2005, 2172-2174), as indicated schematically below:

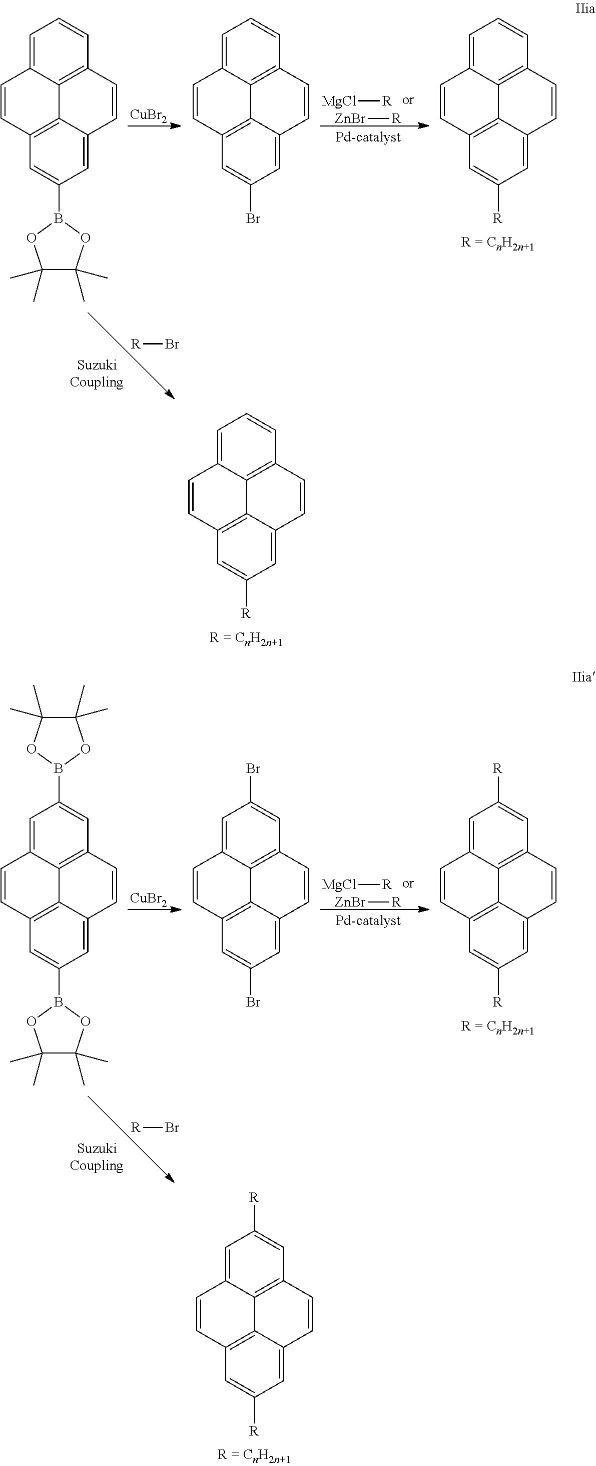

Also branched alkyl chains such as C(n,n) with n from 0 to 16 carbons can be attached following the same strategy as depicted below:

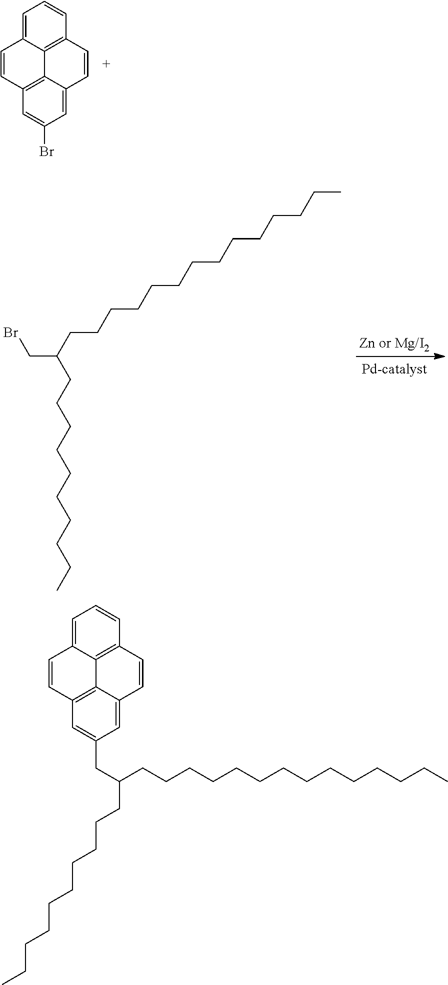

Subsequently, the mono alkylated pyrene can be further processed and polymerized, for example as indicated in the following general scheme:

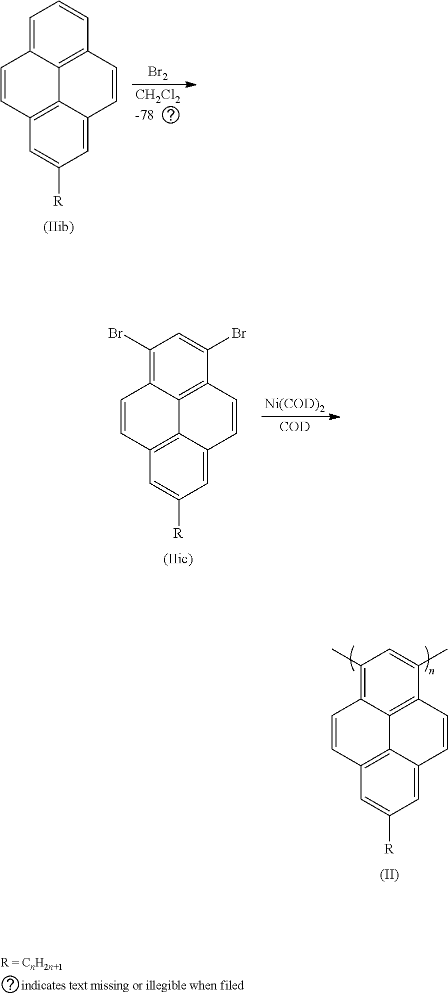

In particular, 1,3-dibromo pyrene monomers of, e.g., formula IIicₜₑᵣₜ (n₁=n₂=0; X¹¹═Br; R¹=tert-butyl) can be produced by reacting the corresponding pyrene of formula IIibₜₑᵣₜ with, e.g. Br₂ or NBS, respectively. Analogous compounds substituted by other halogens, in particular Cl or I, can be prepared by using appropriate starting compounds and analogous reactions or other or other methods known in the art.

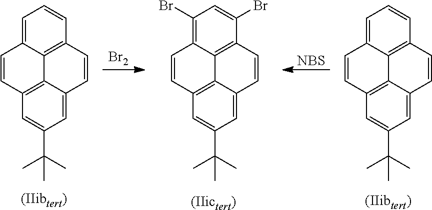

In one embodiment, the polymers according to the invention consist only of one or more type(s) of repeating units of formula I. In a preferred embodiment, the polymers according to the invention consist of precisely one type of repeating unit of formula I (homopolymers). According to the present invention, the term “polymer” comprises polymers in the conventional meaning as well as oligomers, wherein a polymer is a molecule of high relative molecular weight, the structure of which essentially comprises the repetition of units derived, actually or conceptually, from molecules of low relative molecular weight and an oligomer is a molecule of intermediate molecular weight, the structure of which essentially comprises a small plurality of units derived, actually or conceptually, from molecules of lower relative molecular weight. A molecule is regarded as having a high relative molecular weight if it has properties which do not vary significantly with the removal of one or a few of the units. A molecule is regarded as having an intermediate molecular weight if it has properties which do vary significantly with the removal of one or a few of the units.

According to the present invention a homopolymer is a polymer derived from one species of (real, implicit, or hypothetical) monomer. Many polymers are made by the mutual reaction of complementary monomers. These monomers can readily be visualized as reacting to give an “implicit monomer”, the homopolymerisation of which would give the actual product, which can be regarded as a homopolymer. Some polymers are obtained by chemical modification of other polymers, such that the structure of the macromolecules that constitute the resulting polymer can be thought of having been formed by the homopolymerisation of a hypothetical monomer.

Accordingly a copolymer is a polymer derived from more than one species of monomer, e.g. bipolymer, terpolymer, quaterpolymer, etc. The copolymers of the invention may be alternating copolymers, random copolymers or block copolymers. The copolymers may comprise different pyrene monomers and/or other monomers in all possible proportions.

The oligomers of this invention have a weight average molecular weight of <2,000 Daltons. The polymers of this invention preferably have a weight average molecular weight of 2,000 Daltons or greater, especially 2,000 to 2,000,000 Daltons, more preferably 10,000 to 1,000,000 and most preferably 20,000 to 500,000 Daltons. Molecular weights are determined according to gel permeation chromatography using polystyrene or poly(para-phenylene) standards. Typically, a polymer of this invention has more than 20 repeating units, such as 20-50000 repeating units, preferably 20-500 repeating units, whereas an oligomer of the invention has less than 20 repeating units.

In a specific embodiment, the polymer is a copolymer of the following formula (III)

—[[X¹⁰]ₐ-[-T-]_{b}]_{c}-[[—Ar³—]_{d}-[-T-]ₑ]_{f}- (III)

wherein X¹⁰ is a repeating unit of formula I with Ar¹, n₁, Ar², n₂, R₁, R₂, R₃, R₄, R₅, R₆, R₇, and R₈ are as defined above, T is a repeating unit comprising a substituted or unsubstituted aryl or heteroaryl group, in particular as defined in WO06/097419, and Ar³ is a substituted or unsubstituted arylene or heteroarylene group, in particular as defined in WO06/097419, and
a, b, c, d, e, f are numbers or ratios from 0 to 1, and more specifically
a is 1,
b is 0, or 1,
c is 0.005 to 1,
d is 0, or 1,
e is 0, or 1, wherein e is not 1, if d is 0,
f is 0.995 to 0, wherein the sum of c and f is 1.

The repeating units T are in particular selected from the following groups Ta-Tf:

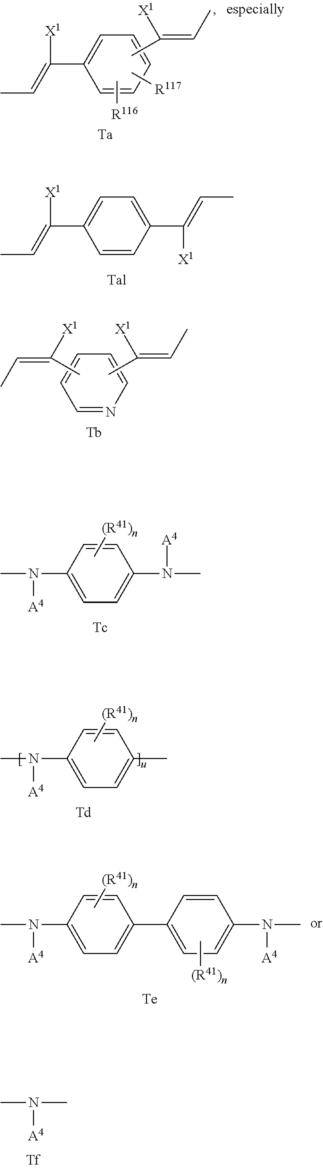

wherein X¹ is a hydrogen atom, or a cyano group,
R¹¹⁶ and R¹¹⁷ are as defined above,
R⁴¹ can be the same or different at each occurence and is Cl, F, CN, N(R⁴⁵)₂, a C₁-C₂₅ alkyl group, a C₄-C₁₈ cycloalkyl group, a C₁-C₂₅ alkoxy group, in which one or more carbon atoms which are not in neighborhood to each other could be replaced by —NR⁴⁵—, —O—, —S—, —C(═O)—O—, or —O—C(═O)—O—, and/or wherein one or more hydrogen atoms can be replaced by F, a C₆-C₂₄ aryl group, or a C₆-C₂₄ aryloxy group, wherein one or more carbon atoms can be replaced by O, S, or N, and/or which can be substituted by one or more non-aromatic groups R⁴¹, or two or more groups R⁴¹ form a ring system;
R⁴⁵ is H, a C₁-C₂₅ alkyl group, a C₄-C₁₈ cycloalkyl group, in which one or more carbon atoms which are not in neighborhood to each other could be replaced by —NR^{45′}—, —O—, —S—, —C(═O)—O—, or —O—C(═O)—O—, and/or wherein one or more hydrogen atoms can be replaced by F, a C₆-C₂₄ aryl group, or a C₆-C₂₄ aryloxy group, wherein one or more carbon atoms can be replaced by O, S, or N, and/or which can be substituted by one or more non-aromatic groups R⁴¹,
R^{45′} is H, a C₁-C₂₅ alkyl group, or a C₄-C₁₈ cycloalkyl group, n can be the same or different at each occurrence and is 0, 1, 2, or 3, especially 0, 1, or 2, very especially 0 or 1, and u is 1, 2, 3, or 4;
A⁴ is a C₆-C₂₄ aryl group, a C₂-C₃₀ heteroaryl group, especially phenyl, naphthyl, anthryl, biphenylyl, 2-fluorenyl, phenanthryl, or perylenyl, which can be substituted by one or more non-aromatic groups R⁴¹.

Preferably, T is a repeating unit of formula Ta, Tb or Tf.

Specifically, Ar³ may be a substituted or unsubstituted benzene, a substituted or unsubstituted naphthalene, a substituted or unsubstituted anthracene, a substituted or unsubstituted diphenylanthracene, a substituted or unsubstituted phenanthrene, a substituted or unsubstituted triphenylene, a substituted or unsubstituted acenaphthene, a substituted or unsubstituted biphenyl, a substituted or unsubstituted fluorene, a substituted or unsubstituted carbazolyl, a substituted or unsubstituted thiophene, substituted or unsubstituted multi-fused thiophenes, a substituted or unsubstituted triazole, a substituted or unsubstituted thiadiazole, a substituted or unsubstituted pyrene, a substituted or unsubstituted triphenylamine, or another chromophore, for example a perylenediimide or perylenemonoimide or higher rylene homologues thereof.

More specifically, Ar³ is selected from the following formulae

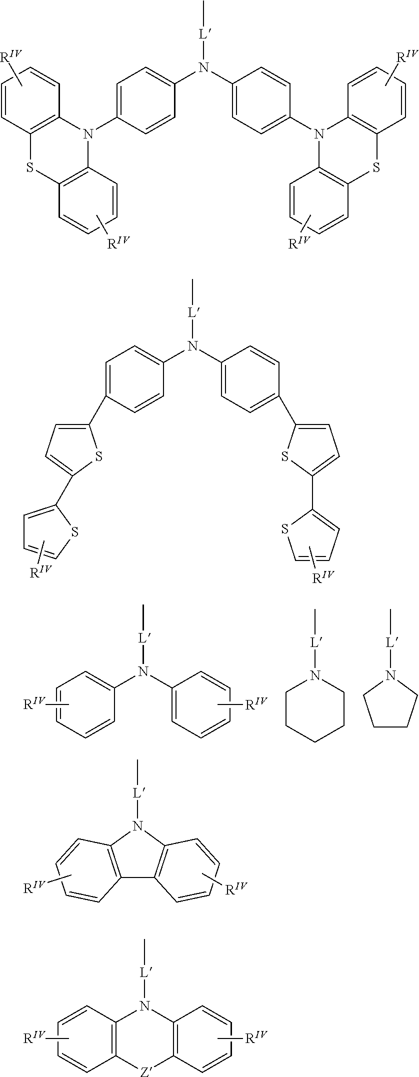

where the variables are each defined as follows:
L′ is a chemical bond or 1,4-phenylene;
Z′ is —O—, —S—, NR^{8′} or —CH₂—, where R^{8′} is C₁-C₁₈-alkyl;
R^{IV} is C₄-C₁₈-alkyl, C₁-C₁₈-alkoxy, (hetero)aryl, or —NR⁵R⁶ with R⁵ and R⁶ independently are as defined above (e.g. pages 5-7).
or from the following formulae:

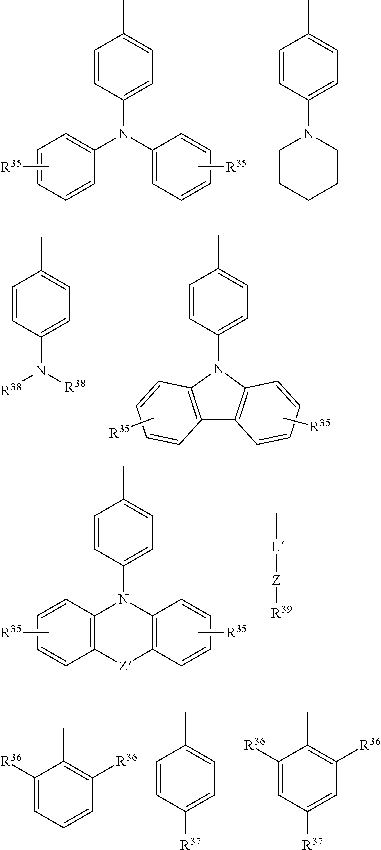

where the variables are each defined as follows:
R³⁵ is C₄-C₁₈-alkyl or C₁-C₁₈-alkoxy;
R³⁶ is C₃-C₈-alkyl, preferably with a secondary carbon atom in the 1-position;
R³⁷ is C₄-C₁₈-alkyl, preferably with a tertiary carbon atom in the 1-position, or R^{9′}R^{10′};
R³⁸ is C₁-C₁₈-alkyl;
R³⁹ is phenyl when L′ is a chemical bond;
C₄-C₁₈-alkyl when L′ is 1,4-phenylene;
L′ is a chemical bond, 1,4-phenylene or 2,5-thienylene;
Z′ is —O—; —S—, —NR^{8′}— or —CH₂—, where R^{8′}, R^{9′} and R^{10′} are C₁-C₁₈-alkyl;

### Z is —O— or —S—;

or from the following formulae

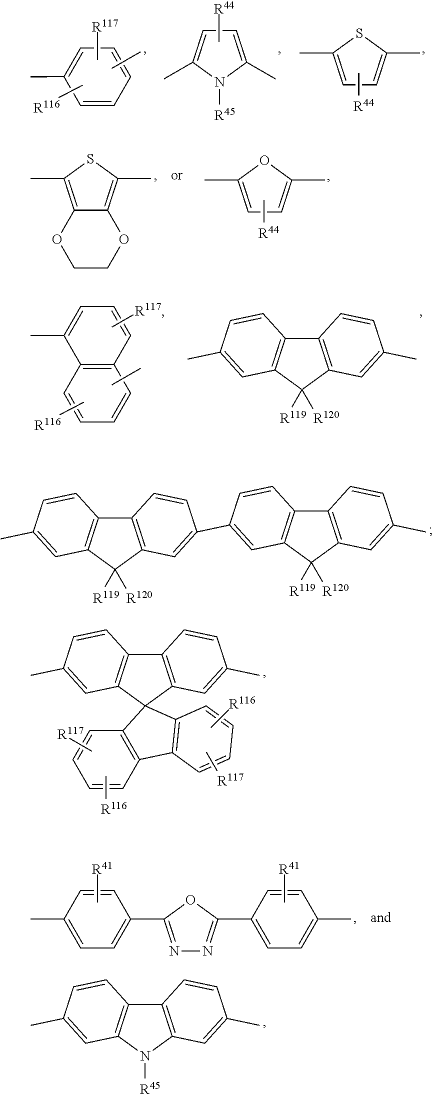

wherein
R⁴⁴ and R⁴¹ are hydrogen, C₁-C₁₈ alkyl, or C₁-C₁₈ alkoxy, and R⁴⁵ is H, C₁-C₁₈ alkyl, or C₁-C₁₈ alkyl which is substituted by E and/or interrupted by D, especially C₁-C₁₈ alkyl which is interrupted by —O—,
R¹¹⁶ and R¹¹⁷ are independently of each other H, halogen, —CN, C₁-C₁₈ alkyl, C₁-C₁₈ alkyl which is substituted by E and/or interrupted by D, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, —C(═O)—R¹²⁷, —C(═O)OR¹²⁷, or —C(═O) NR¹²⁷R¹²⁶,
R¹¹⁹ and R¹²⁰ are independently of each other H, C₁-C₁₈ alkyl, C₁-C₁₈ alkyl which is substituted by E and/or interrupted by D, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, or C7-C25 aralkyl, or R¹¹⁹ and R¹²⁰ together form a group of formula ═CR¹²¹R¹²², wherein R¹²¹ and R¹²² are independently of each other H, C₁-C₁₈ alkyl, C₁-C₁₈ alkyl which is substituted by E and/or interrupted by D, C₆-C₂₄ aryl, C₆-C₂₄ aryl which is substituted by G, or C₂-C₂₀ heteroaryl, or C₂-C₂₀ heteroaryl which is substituted by G,
or R¹¹⁹ and R¹²⁰ together form a five or six membered ring, which optionally can be substituted by C₁-C₁₈ alkyl, C₁-C₁₈ alkyl which is substituted by E and/or interrupted by D, C₆-C₂₄aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, or —C(═O)—R¹²⁷, and
R¹²⁶ and R¹²⁷ are independently of each other H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—,
wherein G, D and E are as defined above.

Polymers of formula III, wherein a=1, b=0, c=1, d=0, e=0, f=0, i.e.—[—X¹⁰—]ₐ (IIIa) are, for example, obtained by nickel coupling reactions as outlined below, especially the Yamamoto reaction.

Copolymers of formula III which involve repeating units of formula I and —Ar³— (a=1, c=0.995 to 0.005, b=0, d=1, e=0, f=0.005 to 0.995), i.e —[—X¹⁰—]_{c}—[—Ar³—]_{f}— (IIIb) also can be obtained by nickel coupling reactions.

Polymerization processes involving only dihalo-functional reactants may be carried out using nickel coupling reactions. One such coupling reaction was described by Colon et al. in J. Pol. Sci., Part A, Polymer Chemistry Edition 28 (1990) 367, and by Colon et al. in J. Org. Chem. 51 (1986) 2627. The reaction is typically conducted in a polar aprotic solvent (e.g., dimethylacetamide) with a catalytic amount of nickel salt, a substantial amount of triphenylphosphine and a large excess of zinc dust. A variant of this process is described by Ioyda et al. in Bull. Chem. Soc. Jpn, 63 (1990) 80, wherein an organo-soluble iodide was used as an accelerator.

Another nickel-coupling reaction was disclosed by Yamamoto in Progress in Polymer Science 17 (1992) 1153, wherein a mixture of dihaloaromatic compounds was treated with an excess amount of nickel (1,5-cyclooctadiene) complex in an inert solvent. All nickel-coupling reactions when applied to reactant mixtures of two or more aromatic dihalides yield essentially random copolymers. Such polymerization reactions may be terminated by the addition of small amounts of water to the polymerization reaction mixture, which will replace the terminal halogen groups with hydrogen groups. Alternatively, a monofunctional aryl halide may be used as a chain-terminator in such reactions, which will result in the formation of a terminal aryl group.

Nickel-coupling polymerizations yield essentially homopolymers or random copolymers comprising units of formula I and units derived from other co-monomers.

Polymers of formula III, wherein a=1, c=1, b=0, d=1, e=0, f=1, i.e. —[—X¹⁰—Ar³—] (IIIc) wherein X¹⁰ and Ar³ are as defined above, can be obtained, for example, by the Suzuki reaction.

The condensation reaction of an aromatic boronate and a halogenide, especially a bromide, commonly referred to as the “Suzuki reaction”, is tolerant of the presence of a variety of organic functional groups as reported by N. Miyaua and A. Suzuki in Chemical Reviews, Vol. 95, pp. 457-2483 (1995). This reaction can be applied to preparing high molecular weight polymers and copolymers. Preferred catalysts are 2-dicyclohexylphosphino-2′,6′-dialkoxybiphenyl/palladium (II) acetates. An especially preferred catalyst is 2-dicyclo-hexylphosphino-2′,6′-di-methoxybiphenyl (sPhos)/palladium (II) acetate.

To prepare polymers corresponding to formula IIIc, a dihalogenide, such as a dibromide or dichloride, especially a dibromide corresponding to formula Br—X¹⁰—Br is reacted with an equimolar amount of a diboronic acid or diboronate corresponding to formula X¹¹—[—Ar³—]—X¹¹, wherein X¹¹ is independently in each occurrence —B(OH)2, —B(OY¹¹)₂,

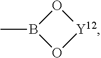

wherein Y¹¹ is independently in each occurrence a C₁-C₁₀ alkyl group and Y¹² is independently in each occurrence a C₂-C₁₀ alkylene group, such as —CY¹³Y¹⁴—CY¹⁵Y¹⁶—, or —CY¹⁷Y¹⁸—CY¹⁹Y²⁰—CY²¹Y²²—, wherein Y¹³, Y¹⁴, Y¹⁵, Y¹⁶, Y¹⁷, Y¹⁸, Y¹⁹, Y²⁰, Y²¹ and Y²² are independently of each other hydrogen, or a C₁-C₁₀ alkyl group, especially —C(CH₃)₂C(CH₃)₂—, or —C(CH₃)₂CH₂C(CH₃)₂—, under the catalytic action of Pd and a phosphine ligand, especially triphenylphosphine. The reaction is typically conducted at about 70° C. to 180° C. in an aromatic hydrocarbon solvent such as toluene. Other solvents such as dimethylformamide and tetrahydrofuran can also be used alone, or in mixtures with an aromatic hydrocarbon. An aqueous base, preferably sodium carbonate, potassium carbonate, K₃PO₄, or bicarbonate, is used as the HBr scavenger. Depending on the reactivities of the reactants, a polymerization reaction may take 2 to 100 hours. Organic bases, such as, for example, tetraalkylammonium hydroxide, and phase transfer catalysts, such as, for example TBAB, can promote the activity of the boron (see, for example, Leadbeater & Marco; Angew. Chem. Int. Ed. Eng. 42 (2003) 1407 and references cited therein). Other variations of reaction conditions are given by T. I. Wallow and B. M. Novak in J. Org. Chem. 59 (1994) 5034-5037; and M. Remmers, M. Schulze, and G. Wegner in Macromol. Rapid Commun. 17 (1996) 239-252. If desired, a monofunctional aryl halide or aryl boronate may be used as a chain-terminator in such reactions, which will result in the formation of a terminal aryl group. It is possible to control the sequencing of the monomeric units in the resulting copolymer by controlling the order and composition of monomer feeds in the Suzuki reaction.

Homopolymers of formula III, wherein a=1, c=1, b=1, d=0, e=0, f=0, i.e. —[X¹⁰-T]-(IIId), wherein X¹⁰ and T are as defined above, can be obtained, for example by the Heck reaction:

Polyphenylenethenylene derivatives and polyphenylenethynylene derivatives can be obtained by polymerization of divinyl or diethinyl compounds with dihalogen compounds by the Heck reaction (R. F. Heck, Palladium Reagents in Organic Synthesis, Academic Press, New York 1985, pp. 179; L. S. Hegedus, Organometalics in Synthesis, Ed. M. Schlosser, Wiley, Chichester, UK 1994, pp. 383; Z. Bao, Y. Chen, R. Cai, L. Yu, Macromolecules 26 (1993) pp. 5281; W.-K. Chan, L. Yu, Macromolecules 28 (1995) pp. 6410; A. Hilberer, H.-J. Brouwer, B.-J. van der Scheer, J. Wildeman, G. Hadziioannou, Macromolecules 1995, 28, 4525) and the Sonogaschira reaction (Dmitri Gelman and Stephen L. Buchwald, Angew. Chem. Int. Ed. 42 (2003) 5993-5996; Rik R. Tykwinski, Angew. Chem. 115 (2003) 1604-1606; Jason M. Nolan and Daniel L. Comins, J. Org. Chem. 68 (2003) 3736-3738; Jiang Cheng et al., J. Org. Chem. 69 (2004) 5428-5432; Zolta'n Nova'k et al., Tetrahedron 59 (2003) 7509-7513):

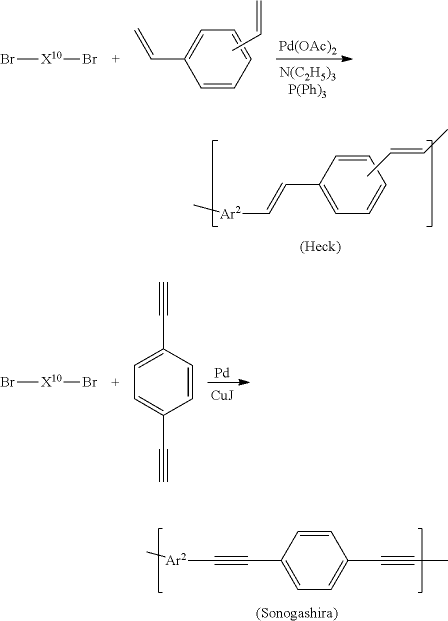

The Sonogashira reaction is done in the presence a copper (I) catalyst, and/or palladium(0), such as, for example, tetrakis (triphenyl-phosphine) palladium(0), optionally in a solvent, such as toluene, dimethyl formamide, or dimethyl sulfoxide, and optionally a base, such as sodium hydride, potassium carbonate, sodium carbonate, or an amine base, such as piperidine. With special palladium catalysts the copper catalyst is not required (Angew. Chem. 2007, 119, 850-888). The reaction time and temperature depends on the starting materials and reaction conditions. Usually the dibromo-compound is reacted with the alkyne at a temperature of from 50° C. to 100° C., especially 60 to 80° C., for 1 h to 48 h hours. This reaction, referred to as an Sonogashira reaction (Pd/Cu-catalyzed cross-coupling of organohalides with terminal alkynes), Cadiot-Chodkiewicz coupling or Castro-Stephens reaction (the Castro-Stephens coupling uses stoichiometric copper, whereas the Sonogashira variant uses catalytic palladium and copper), is described by Sonogashira K.; Tohda, Y.; Hagihara, N. Tetrahedron Lett. 1975, 4467; Richard Heck (discovered the same transformation using palladium but without the use of copper) J. Organomet. Chem. 1975, 93, 259; McCrindle, R.; Ferguson, G.; Arsenaut, G. J.; McAlees, A. J.; Stephenson, D. K. J. Chem. Res. (S) 1984, 360; Sakamoto, T.; Nagano, T.; Kondo, Y.; Yamanaka, H. Chem. Pharm. Bull. 1988, 36, 2248; Rossi, R. Carpita, A.; Belina, F. Org. Prep. Proc. Int. 1995, 27, 129; Ernst, A.; Gobbi, L.; Vasella, A. Tetrahedron Lett. 1996, 37, 7959; Campbell, I. B. In Organocopper Reagents; Taylor, R. J. K. Ed.; IRL Press: Oxford, UK, 1994, 217. (Review); Hundermark, T.; Littke, A.; Buchwald, S. L.; Fu, G. C. Org. Lett. 2000, 2, 1729; Dai, W.-M.; Wu, A. Tetrahedron Lett. 2001, 42, 81; Alami, M.; Crousse, B.; Ferri, F. J. Organomet. Chem. 2001, 624, 114; Bates, R. W.; Boonsombat, J. J. Chem. Soc., Perkin Trans. 1 2001, 654; Batey, R. A.; Shen, M.; Lough, A. J. Org. Lett. 2002, 4, 1411; Balova, I. A.; Morozkina, S, N.; Knight, D. W.; Vasilevsky, S. F. Tetrahedron Lett. 2003, 44, 107; Garcia, D.; Cuadro, A. M.; Alvarez-Builla, J.; Vaquero, J. J. Org. Lett. 2004, 6, 4175; Li, P.; Wang, L.; Li, H. Tetrahedron 2005, 61, 8633, Lemhadri, M.; Doucet, H.; Santelli, M. Tetrahedron 2005, 61, 9839, Angew. Chem. 2007, 119, 8632-8635, Angew. Chem. 2006, 118, 6335-6339, J. Am. Chem. Soc. 2005, 127, 9332-9333, and Adv. Mater. 2007, 19, 1234-1238.

(Random) copolymers of formula III, wherein a is 1, b is 1, c is 0.005 to 0.995, d is 1, e is 1, f is 0.995 to 0.005, wherein the sum of c and f is 1, can also be obtained by the Heck reaction: —[[X¹⁰]ₐ-[-T-]_{b}]_{c}-[[—Ar³—]_{d}-[-T-]ₑ]_{f}-(IIIe), wherein a, b, c, d, e, f, X¹⁰, Ar³ and T are as defined above.

The polymers containing groups of formulas (I) may be prepared by any suitable process, but are preferably prepared by the processes described above.

The polymers of the present invention can optionally comprise end moieties E¹, wherein E¹ is an aryl moiety which may optionally be substituted with a reactive group capable of undergoing chain extension or crosslinking, or a tri(C₁-C₁₈)alkylsiloxy group. As used herein, a reactive group capable of undergoing chain extension or crosslinking refers to any group which is capable of reacting with another of the same group or another group so as to form a link to prepare polymers. Preferably, such reactive group is a hydroxy, glycidyl ether, acrylate ester, methacrylate ester, ethenyl, ethynyl, maleimide, naphthimide, oxetane, trifluorovinyl ether moiety or a cyclobutene moiety fused to the aromatic ring of E¹.

The polymers of the present invention, where E¹ are reactive groups as defined above, are capable of crosslinking to form solvent resistant, heat-resistant films at 100° C. or more, more preferably at 150° C. or more. Preferably, such crosslinking occurs at 350° C. or less, more preferably 300° C. or less and most preferably 250° C. or less. The crosslinkable polymers of the invention are stable at 100° C. or more and more preferably 150° C. or more. “Stable” as used herein means that such polymers do not undergo crosslinking or polymerization reactions at or below the stated temperatures. If a crosslinkable material is desired, E¹ is preferably a vinylphenyl, an ethynylphenyl, or 4-(or 3-)benzocyclobutenyl radical. In another embodiment, E¹ is selected from a group of phenolic derivatives of the formula —C₆H₄—O—Y, wherein Y is

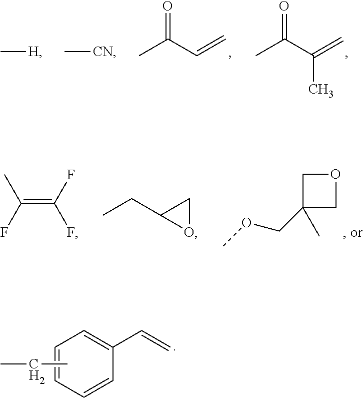

If desired, the cross-linkable groups can be present in other parts of the polymer chain. For example, one of the substituents of the co-monomer T may be a crosslinkable group E¹.

The end-capping agent E¹-X¹² (E¹ is as defined above and X¹² is either Cl or Br) is incorporated into the polymers of the present invention under the condition in which the resulting polymers are substantially capped by the reactive group E¹. The reactions useful for this purpose are the nickel-coupling, Heck reactions and Suzuki reactions described above.

The average degree of polymerization is controlled by the mole ratio of monomers to end-capping agent.

### DEFINITIONS

Halogen is fluorine, chlorine, bromine and iodine.

C₁-C₁₈ alkyl (C₁-C₂₅ alkyl) is typically linear or branched, where possible. Examples are methyl, ethyl, n-propyl, isopropyl, n-butyl, sec-butyl, isobutyl, tert-butyl, n-pentyl, 2-pentyl, 3-pentyl, 2,2-dimethylpropyl, 1,1,3,3-tetramethylpentyl, n-hexyl, 1-methylhexyl, 1,1,3,3,5,5-hexamethylhexyl, n-heptyl, isoheptyl, 1,1,3,3-tetra-methylbutyl, 1-methylheptyl, 3-methylheptyl, n-octyl, 1,1,3,3-tetramethylbutyl and 2-ethylhexyl, n-nonyl, decyl, undecyl, dodecyl, tridecyl, tetradecyl, pentadecyl, hexadecyl, heptadecyl, octadecyl, eicosyl, heneicosyl, docosyl, tetracosyl or pentacosyl. C₁-C₈ alkyl is typically methyl, ethyl, n-propyl, isopropyl, n-butyl, sec.-butyl, isobutyl, tert-butyl, n-pentyl, 2-pentyl, 3-pentyl, 2,2-dimethyl-propyl, n-hexyl, n-heptyl, n-octyl, 1,1,3,3-tetramethylbutyl and 2-ethylhexyl. C₁-C₄ alkyl is typically methyl, ethyl, n-propyl, isopropyl, n-butyl, sec-butyl, isobutyl, tert-butyl.

C₁-C₂₅ alkoxy (C₁-C₁₈ alkoxy) groups are straight-chain or branched alkoxy groups, e.g. methoxy, ethoxy, n-propoxy, isopropoxy, n-butoxy, sec-butoxy, tert-butoxy, amyloxy, isoamyloxy or tert-amyloxy, heptyloxy, octyloxy, isooctyloxy, nonyloxy, decyloxy, undecyloxy, dodecyloxy, tetradecyloxy, pentadecyloxy, hexadecyloxy, heptadecyloxy and octadecyloxy. Examples of C₁-C₈ alkoxy are methoxy, ethoxy, n-propoxy, isopropoxy, n-butoxy, sec.-butoxy, isobutoxy, tert.-butoxy, n-pentyloxy, 2-pentyloxy, 3-pentyloxy, 2,2-dimethylpropoxy, n-hexyloxy, n-heptyloxy, n-octyloxy, 1,1,3,3-tetramethylbutoxy and 2-ethylhexyloxy. Examples of C₁-C₄ alkoxy are methoxy, ethoxy, n-propoxy, isopropoxy, n-butoxy, sec.-butoxy, isobutoxy, tert.-butoxy. The term “alkylthio group” means the same groups as the alkoxy groups, except that the oxygen atom of the ether linkage is replaced by a sulfur atom.

C₂-C₂₅ alkenyl (C₂-C₁₈ alkenyl) groups are straight-chain or branched alkenyl groups, such as e.g. vinyl, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, isododecenyl, n-dodec-2-enyl or n-octadec-4-enyl.

C₂-C₂₄ alkynyl (C₂-C₁₈ alkynyl) is straight-chain or branched and preferably C₂-C₈ alkynyl, which may be unsubstituted or substituted, such as, for example, ethynyl, 1-propyn-3-yl, 1-butyn-4-yl, 1-pentyn-5-yl, 2-methyl-3-butyn-2-yl, 1,4-pentadiyn-3-yl, 1,3-pentadiyn-5-yl, 1-hexyn-6-yl, cis-3-methyl-2-penten-4-yn-1-yl, trans-3-methyl-2-penten-4-yn-1-yl, 1,3-hexadiyn-5-yl, 1-octyn-8-yl, 1-nonyn-9-yl, 1-decyn-10-yl, or 1-tetracosyn-24-yl.

C₁-C₁₈ perfluoroalkyl, especially C₁-C₄ perfluoroalkyl, is a branched or unbranched radical such as for example —CF₃, —CF₂CF₃, —CF₂CF₂CF₃, —CF (CF₃)₂, —(CF₂)₃CF₃, and —C(CF₃)₃.

The terms “haloalkyl, haloalkenyl and haloalkynyl” mean groups given by partially or wholly substituting the above-mentioned alkyl group, alkenyl group and alkynyl group with halogen, such as trifluoromethyl etc. The “aldehyde group, ketone group, ester group, carbamoyl group and amino group” include those substituted by an alkyl group, a cycloalkyl group, an aryl group, an aralkyl group or a heterocyclic group, wherein the alkyl group, the cycloalkyl group, the aryl group, the aralkyl group and the heterocyclic group may be unsubstituted or substituted. The term “silyl group” means a group of formula —SiR⁶²R⁶³R⁶⁴, wherein R⁶², R⁶³ and R⁶⁴ are independently of each other a C₁-C₈ alkyl group, in particular a C₁-C₄ alkyl group, a C₆-C₂₄ aryl group or a C₇-C₁₂ aralkylgroup, such as a trimethylsilyl group.

The term “cycloalkyl group” refers typically to C₄-C₁₈ cycloalkyl, especially C₅-C₁₂ cycloalkyl, such as cyclopentyl, cyclohexyl, cycloheptyl, cyclooctyl, cyclononyl, cyclodecyl, cycloundecyl, cyclododecyl, preferably cyclopentyl, cyclohexyl, cycloheptyl, or cyclooctyl, which may be unsubstituted or substituted. The term “cycloalkenyl group” means an unsaturated alicyclic hydrocarbon group containing one or more double bonds, such as cyclopentenyl, cyclopentadienyl, cyclohexenyl and the like, which may be unsubstituted or substituted. The cycloalkyl group, in particular a cyclohexyl group, can be condensed one or two times by phenyl which can be substituted one to three times with C₁-C₄-alkyl, halogen and cyano. Examples of such condensed cyclohexyl groups are:

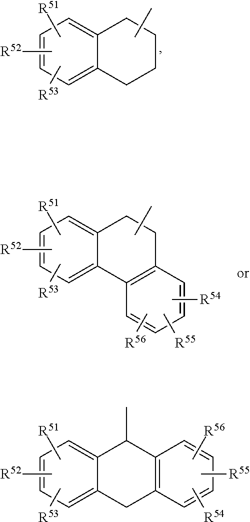

in particular

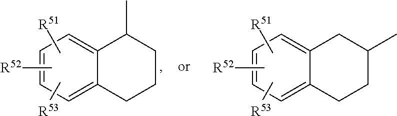

wherein R⁵¹, R⁵², R⁵³, R⁵⁴, R⁵⁵ and R⁵⁶ are independently of each other C₁-C₈ alkyl, C₁-C₈ alkoxy, halogen and cyano, in particular hydrogen.

Aryl is usually C₆-C₃₀ aryl, preferably C₆-C₂₄ aryl (C₆-C₁₈ aryl), which optionally can be substituted, such as, for example, phenyl, 4-methylphenyl, 4-methoxyphenyl, naphthyl, especially 1-naphthyl, or 2-naphthyl, biphenylyl, terphenylyl, pyrenyl, 2- or 9-fluorenyl, phenanthryl, anthryl, tetracyl, pentacyl, hexacyl, quaderphenylyl, or rylene derivatives, such as perylenes, terrylenes or higher rylenes which may be unsubstituted or substituted.

The term “aralkyl group” refers typically to C₇-C₂₅ aralkyl, such as benzyl, 2-benzyl-2-propyl, b-phenyl-ethyl, a,a-dimethylbenzyl, w-phenyl-butyl, w,w-dimethyl-w-phenyl-butyl, w-phenyldodecyl, w-phenyl-octadecyl, w-phenyl-eicosyl or w-phenyl-docosyl, preferably C₇-C₁₈aralkyl such as benzyl, 2-benzyl-2-propyl, b-phenyl-ethyl, a,a-dimethylbenzyl, w-phenyl-butyl, w,w-dimethyl-w-phenyl-butyl, w-phenyl-dodecyl or w-phenyl-octadecyl, and particularly preferred C₇-C₁₂ aralkyl such as benzyl, 2-benzyl-2-propyl, b-phenyl-ethyl, a,a-dimethylbenzyl, w-phenyl-butyl, or w,w-dimethyl-w-phenyl-butyl, in which both the aliphatic hydrocarbon group and aromatic hydrocarbon group may be unsubstituted or substituted.

The term “aryl ether group” refers typically to a C₆₋₂₄ aryloxy group, that is to say O—C₆-C₂₄ aryl, such as, for example, phenoxy or 4-methoxyphenyl. The term “aryl thioether group” means typically a C₆₋₂₄ arylthio group, that is to say S—C₆-₂₄-aryl, such as, for example, phenylthio or 4-methoxyphenylthio. The term “carbamoyl group” refers typically to a C₁-c₁₈ carbamoyl radical, preferably C₁-₈ carbamoyl radical, which may be unsubstituted or substituted, such as, for example, carbamoyl, methylcarbamoyl, ethylcarbamoyl, n-butylcarbamoyl, tert-butylcarbamoyl, dimethylcarbamoyloxy, morpholinocarbamoyl or pyrrolidinocarbamoyl.

The terms “aryl” and “alkyl” in alkylamino groups, dialkylamino groups, alkylarylamino groups, arylamino groups and diarylgroups typically refer to C₁-C₂₅ alkyl and C₆-C₂₄ aryl, respectively.

Alkylaryl refers to alkyl-substituted aryl radicals, especially C₇-C₁₂ alkylaryl. Examples are tolyl, such as 3-methyl-, or 4-methylphenyl, or xylyl, such as 3,4-dimethylphenyl, or 3,5-dimethylphenyl.

Heteroaryl is typically C₂-C₂₆ heteroaryl (C₂-C₂₀ heteroaryl), i.e. a ring with five to seven ring atoms or a condensed ring system, wherein nitrogen, oxygen or sulfur are the possible hetero atoms, and is typically an unsaturated heterocyclic group with five to 30 atoms having at least six conjugated p-electrons such as thienyl, benzo[b]thienyl, dibenzo[b,d]-thienyl, thianthrenyl, furyl, furfuryl, 2H-pyranyl, benzofuranyl, isobenzofuranyl, dibenzofuranyl, phenoxythienyl, pyrrolyl, imidazolyl, pyrazolyl, pyridyl, bipyridyl, triazinyl, pyrimidinyl, pyrazinyl, pyridazinyl, indolizinyl, isoindolyl, indolyl, indazolyl, purinyl, quinolizinyl, chinolyl, isochinolyl, phthalazinyl, naphthyridinyl, chinoxalinyl, chinazolinyl, cinnolinyl, pteridinyl, carbazolyl, carbolinyl, benzotriazolyl, benzoxazolyl, phenanthridinyl, acridinyl, pyrimidinyl, phenanthrolinyl, phenazinyl, isothiazolyl, phenothiazinyl, isoxazolyl, furazanyl or phenoxazinyl, which can be unsubstituted or substituted. Possible substituents of the above-mentioned groups are C₁-C₈ alkyl, a hydroxyl group, a mercapto group, C₁-C₈ alkoxy, C₁-C₈ alkylthio, halogen, halo-C₁-C₈ alkyl, a cyano group, an aldehyde group, a ketone group, a carboxyl group, an ester group, a carbamoyl group, an amino group, a nitro group or a silyl group, especially C₁-C₈ alkyl, C₁-C₈ alkoxy, C₁-C₈ alkylthio, halo-C₁-C₈ alkyl, or a cyano group.

If a substituent, such as, for example R⁶ occurs more than one time in a group, it can be different in each occurrence.

The wording “substituted by G” means that one or more, especially one to three substituents G might be present.

As described above, the aforementioned groups may be substituted by E and/or, if desired, interrupted by D. Interruptions are of course possible only in the case of groups containing at least 2 carbon atoms connected to one another by single bonds; C₆-C₁₈ aryl is not interrupted; interrupted arylalkyl or alkylaryl contains the unit D in the alkyl moiety. A C₁-C₁₈ alkyl group substituted by one or more E and/or interrupted by one or more units D is, for example, (CH₂CH₂O)₁₋₉—R^{x}, where R^{x} is H or C₁-C₁₀ alkyl or C₂-C₁₀ alkanoyl (e.g. CO—CH(C₂H₅) C₄H₉), CH₂—CH(OR^{y}′)—CH₂—O—R^{y}, where R^{y} is C₁-C₁₈ alkyl, C₅-C₁₂ cycloalkyl, phenyl, C₇-C₁₅ phenylalkyl, and R^{y}′ embraces the same definitions as R^{y} or is H; C₁-C₈ alkylene-COO—R^{z}, e.g. CH₂COOR^{z}, CH(CH₃) COOR^{z}, C(CH₃)₂COOR^{z}, where R^{z} is H, C₁-C₁₈ alkyl, (CH₂CH₂O)₁₋₉—R^{x}, and R^{x} embraces the definitions indicated above; CH₂CH₂—O—CO—CH═CH₂: CH₂CH(OH)CH₂—O—CO—C(CH₃)═CH₂.

Preferred arylene radicals are 1,4-phenylene, 2,5-tolylene, 1,4-naphthylene, 1,9 anthracylene, 2,7-phenanthrylene and 2,7-dihydrophenanthrylene.

Preferred heteroarylene radicals are 2,5-pyrazinylene, 3,6-pyridazinylene, 2,5-pyridinylene, 2,5-pyrimidinylene, 1,3,4-thiadiazol-2,5-ylene, 1,3-thiazol-2,4-ylene, 1,3-thiazol-2,5-ylene, 2,4-thiophenylene, 2,5-thiophenylene, 1,3-oxazol-2,4-ylene, 1,3-oxazol-2,5-ylene and 1,3,4-oxadiazol-2,5-ylene, 2,5-indenylene and 2,6-indenylene.

The polymers according to the invention can be worked up by known methods which are familiar to the person skilled in the art, as described, for example, in D. Braun, H. Cherdron, H. Ritter, Praktikum der makromolekularen Stoffe, 1st Edn., Wiley VCH, Weinheim 1999, p. 68-79 or R. J. Young, P. A. Lovell, Introduction to Polymers, Chapman & Hall, London 1991.

For example, the reaction mixture can be filtered, diluted with aqueous acid, extracted and the crude product obtained after drying and stripping-off of the solvent can be further purified by reprecipitation from suitable solvents with addition of precipitants. Residual palladium can be removed by using activated carbon, chromatography etc. Advantageously, the residual palladium could be reduced to <3 ppm by washing the crude organic solvent layer containing the polymer with an aqueous solution of L-cysteine at room temperature to the boiling point of the organic solvent, especially by washing a toluene layer containing the polymer with an aqueous solution of L-cysteine at 85 to 90° C., optionally followed by washing with a solution of L-cysteine and sodium thiosulfate at 78 to 82° C. (Mahavir Prashad, Yugang Liu, Oljan Repicoe, Adv. Synth. Catal. 2003, 345, 533-536; Christine E. Garrett, Kapa Prasad, Adv. Synth. Catal. 2004, 346, 889-900). Additionally the Pd can be removed by washing the polymer with an aqueous NaCN solution as described in U.S. Pat. No. B 6,956,095. Polymeranalogous reactions can subsequently be carried out for further functionalization of the polymer. Thus, for example, terminal halogen atoms can be removed reductively by reduction with, for example, LiAlH₄ (see, for example, J. March, Advanced Organic Chemistry, 3rd Edn. McGraw-Hill, p. 510).

A further aspect of the present invention is related to polymer blends containing 1 to 99 percent of at least one component containing polymers comprising a unit of formula I. The remainder 1 to 99 percent of the blend is composed of one or more polymeric materials selected from chain growth polymers such as polystyrene, polybutadiene, poly(methyl methacrylate), and poly(ethylene oxide); step-growth polymers such as phenoxy resins, polycarbonates, polyamides, polyesters, polyurethanes, and polyimides; and crosslinked polymers such as crosslinked epoxy resins, crosslinked phenolic resins, crosslinked acrylate resins, and crosslinked urethane resins. Examples of these polymers may be found in Preparative Methods of Polymer Chemistry, W. R. Sorenson and T. W. Campbell, Second Edition, Interscience Publishers (1968). Also usable in the blends are conjugated polymers such as poly(phenylene vinylene), substituted poly(phenylene vinylene)s, substituted polyphenylenes and polythiophenes. Examples of these conjugated polymers are given by Greenham and Friend in Solid State Physics, Vol. 49, pp. 1-149 (1995).

Another aspect of the invention is the films formed from the polymers of the invention. Such films can be used in polymeric light-emitting diodes (PLEDs). Preferably, such films are used as emitting layers. These films may also be used as protective coatings for electronic devices and as fluorescent coatings. The thickness of the coating or film is dependent upon the ultimate use. Generally, such thickness can be from 0.01 to 200 microns. In that embodiment wherein the coating is used as a fluorescent coating, the coating or film thickness is from 10 to 200 microns. In that embodiment where the coatings are used as electronic protective layers, the thickness of the coating can be from 5 to 20 microns. In that embodiment where the coatings are used in a polymeric light-emitting diode, the thickness of the layer formed is 0.01 to 0.5 microns. The polymers of the invention form good pinhole- and defect-free films. Such films can be prepared by means well known in the art including spin-coating, spray-coating, dip-coating and roller-coating. Such coatings are prepared by a process comprising applying a composition to a substrate and exposing the applied composition to conditions such that a film is formed. The conditions which form a film depend upon the application technique. Preferably, the solution contains from 0.1 to 10 weight percent of the polymers. This composition is applied to the appropriate substrate by the desired method and the solvent is allowed to evaporate. Residual solvent may be removed by vacuum and/or by heat-drying. The films are preferably substantially uniform in thickness and substantially free of pinholes. In another embodiment, the polymers may be partially cured. This is known as B-staging.

A further embodiment of the present invention is directed to an electronic device or a component therefore, comprising a substrate and a polymer according to the present invention.

In such a device the polymers according to the present invention are used as electroluminescent material. For the purposes of the present invention, the term “electroluminescent material” is intended to mean materials which can be used as or in an active layer in an electroluminescent device. The term “active layer” means that the layer is capable of emitting light (light-emitting layer) on application of an electric field and/or that it improves the injection and/or transport of the positive and/or negative charges (charge injection or charge transport layer). The invention therefore also relates to the use of the polymers according to the invention as electroluminescent material. The invention furthermore relates to an electroluminescent material which comprises the polymers according to the invention.

Electroluminescent devices are used, for example, as self-illuminating display elements, such as control lamps, alphanumeric displays, signs and in opto-electronic couplers.

A device according to the present invention may be prepared in accordance with the disclosure of WO99/48160, the contents of which are incorporated by reference. Polymers according to the present invention may be present in the device as the sole light emitting polymer or as a component in a blend further comprising hole and/or electron transporting polymers. Alternatively, the device may comprise distinct layers of a polymer of the present invention, a hole transporting polymer and/or an electron transporting polymer.

In one embodiment the electronic device comprises an electro-luminescent device, which comprises

(a) a charge injecting layer for injecting positive charge carriers,
(b) a charge injecting layer for injecting negative charge carriers,
(c) a light-emissive layer located between the layers (a) and (b) comprising a polymer according to the present invention.

The layer (a) may be a positive charge carrier transport layer which is located between the light emissive layer (c) and an anode electrode layer, or may be an anode electrode layer.

The layer (b) may be a negative charge carrier transport layer which is located between the light emissive layer (c) and an cathode electrode layer, or may be an cathode electrode layer. Optionally, an organic charge transport layer can be located between the light emissive layer (c) and one of the charge carrier injecting layers (a) and (b).

The EL device emits light in the visible electro-magnetic spectrum between 400 nm and 780 nm, preferably between 430 nm and 470 nm for a blue color, preferably between 520 nm and 560 nm for a green color, preferably between 600 nm and 650 nm for a red color. By incorporating specific repeating units in the backbone of the polymer the emission can be even shifted to the near infrared (NIR, >780 nm).

It will be evident that the light emissive layer may be formed from a blend or mixture of materials including one or more polymers according to the present invention, and optionally further different polymers. The further different polymers may be so-called hole transport polymers (i.e. to improve the efficiency of hole transport to the light-emissive material) or electron-transport polymers (i.e. to improve the efficiency of electron transport to the light emissive material). Preferably, the blend or mixture would comprise at least 0.1% by weight of a polymer according to the present invention, preferably at least 0.5% by weight, more preferably at least 1% by weight.

An organic EL device typically consists of an organic film sandwiched between an anode and a cathode such that when a positive bias is applied to the device, holes are injected into the organic film from the anode, and electrons are injected into the organic film from the cathode.

The combination of a hole and an electron may give rise to an exciton, which may undergo radiative decay to the ground state by liberating a photon. In practice the anode is commonly an mixed oxide of tin and indium for its conductivity and transparency. The mixed oxide (ITO) is deposited on a transparent substrate such as glass or plastic so that the light emitted by the organic film may be observed. The organic film may be the composite of several individual layers each designed for a distinct function. Since holes are injected from the anode, the layer next to the anode needs to have the functionality of transporting holes.

Similarly, the layer next to the cathode needs to have the functionality of transporting electrons. In many instances, the hole-(electron) transporting layer also acts as the emitting layer. In some instances one layer can perform the combined functions of hole and electron transport and light emission. The individual layers of the organic film may be all polymeric in nature or combinations of films of polymers and films of small molecules deposited by thermal evaporation. The total thickness of the organic film be less than 1000 nanometers (nm), especially less than 500 nm. It is preferred that the total thickness be less than 300 nm, while it is more preferred that the thickness of the active layer is in the range from 40-160 nm.

The ITO-glass, which serves as the substrate and the anode, may be used for coating after the usual cleaning with detergent, organic solvents and UV-ozone treatment. It may also be first coated with a thin layer of a conducting substance to facilitate hole injection. Such substances include copper phthalocyanine, polyaniline (PANI) and poly(3,4-ethylenedioxythiophene) (PEDOT); the last two in their (doped) conductive forms, doped, for example, with FeCl₃ or Na₂S₂O₈. They contain poly(styrenesulfonic acid) (PSS) as counter-ion to ensure water solubility. It is preferred that the thickness of this layer be 200 nm or less; it is more preferred that the thickness be 100 nm or less.

In the cases where a hole-transporting layer is used, the polymeric arylamines described in U.S. Pat. No. 5,728,801, may be used. Other known hole-conducting polymers, such as polyvinylcarbazole, may also be used. The resistance of this layer to erosion by the solution of the copolymer film which is to be applied next is obviously critical to the successful fabrication of multi-layer devices. The thickness of this layer may be 500 nm or less, preferably 300 nm or less, most preferably 150 nm or less.

In the case where an electron-transporting layer is used, it may be applied either by thermal evaporation of low molecular weight materials or by solution coating of a polymer with a solvent that would not cause significant damage to the underlying film.

Examples of low molecular weight materials include the metal complexes of 8-hydroxyquinoline (as described by Burrows et al. in Appl. Phys. Lett. 64 (1994) 2718-2720), metallic complexes of 10-hydroxybenzoquinoline (as described by Hamada et al. in Chem. Lett. (1993) 906-906), 1,3,4-oxadiazoles (as described by Hamada et al. in Optoelectronics-Devices and Technologies 7 (1992) 83-93), 1,3,4-triazoles (as described by Kido et al. in Chem. Lett. (1996) 47-48), and dicarboximides of perylene (as described by Yoshida et al. in Appl. Phys. Lett. 69 (1996) 734-736).

Polymeric electron-transporting materials are exemplified by 1,3,4-oxadiazole-containing polymers (as described by Li et al. in J. Chem. Soc. (1995) 2211-2212, by Yang and Pei in J. Appl. Phys. 77 (1995) 4807-4809), 1,3,4-triazole-containing polymers (as described by Strukelj et al. in Science 267 (1995) 1969-1972), quinoxaline-containing polymers (as described by Yamamoto et al. in Jpn. J. Appl. Phys. 33 (1994) L250-L253, O'Brien et al. in Synth. Met. 76 (1996) 105-108), and cyano-PPV (as described by Weaver et al. in Thin Solid Films 273 (1996) 39-47). The thickness of this layer may be 500 nm or less, preferably 300 nm or less, most preferably 150 nm or less.

The cathode material may be deposited either by thermal evaporation or by sputtering. The thickness of the cathode may be from 1 nm to 10,000 nm, preferably 5 nm to 500 nm.

OLEDs made according to the present invention may include phosphorescent dopants dispersed in the device's emissive layer, capable of achieving internal quantum efficiencies approaching 100%. As used herein, the term “phosphorescence refers to emission from a triplet excited state of an organic or metal-organic molecule. High efficiency organic light emitting devices using phosphorescent dopants have been demonstrated using several different conducting host materials (M. A. Baldo et al., Nature, Vol 395, 151 (1998), C. Adachi et al., Appl. Phys. Lett., Vol. 77, 904 (2000)).

In a preferred embodiment, the electroluminescent device comprises at least one hole-transporting polymer film and a light-emitting polymer film comprised of the polymer of the invention, arranged between an anode material and a cathode material such that under an applied voltage, holes are injected from the anode material into the hole-transporting polymer film and electrons are injected from the cathode material into the light-emitting polymer films when the device is forward biased, resulting in light emission from the light-emitting layer.

In another preferred embodiment, layers of hole-transporting polymers are arranged so that the layer closest to the anode has the lower oxidation potential, with the adjacent layers having progressively higher oxidation potentials. By these methods, electroluminescent devices having relatively high light output per unit voltage may be prepared.

The term “hole-transporting polymer film” as used herein refers to a layer of a film of a polymer which when disposed between two electrodes to which a field is applied and holes are injected from the anode, permits adequate transport of holes into the emitting polymer. Hole-transporting polymers typically are comprised of triarylamine moieties.

The term “light emitting polymer film” as used herein refers to a layer of a film of a polymer whose excited states can relax to the ground state by emitting photons, preferably corresponding to wavelengths in the visible range. The term “anode material” as used herein refers to a semitransparent, or transparent, conducting film with a work function between 4.5 electron volts (eV) and 5.5 eV. Examples are gold, silver, copper, aluminum, indium, iron, zinc, tin, chromium, titanium, vanadium, cobalt, nickel, lead, manganese, tungsten and the like, metallic alloys such as magnesium/copper, magnesium/silver, magnesium/aluminum, aluminum/indium and the like, semiconductors such as Si, Ge, GaAs and the like, metallic oxides such as indium-tin-oxide (“ITO”), ZnO and the like, metallic compounds such as CuI and the like, and furthermore, electroconducting polymers such polyacetylene, polyaniline, polythiophene, polypyrrole, polyparaphenylene and the like. Oxides and mixed oxides of indium and tin, and gold are preferred. Most preferred is ITO, especially ITO on glass, or on a plastics material, such as polyester, for example polyethylene terephthalate (PET), as substrate.

The term “cathode material” as used herein refers to a conducting film with a work function between 2.0 eV and 4.5 eV. Examples are alkali metals, earth alkaline metals, group 13 elements, silver, and copper as well as alloys or mixtures thereof such as sodium, lithium, potassium, calcium, lithium fluoride (LiF), sodium-potassium alloy, magnesium, barium, magnesium-silver alloy, magnesium-copper alloy, magnesium-aluminum alloy, magnesiumindium alloy, aluminum, aluminum-aluminum oxide alloy, aluminum-lithium alloy, indium, calcium, and materials exemplified in EP-A 499,011, such as electroconducting polymers e.g. polypyrrole, polythiophene, polyaniline, polyacetylene etc. Preferably lithium, barium, calcium, magnesium, indium, silver, aluminum, or blends and alloys of the above are used. In the case of using a metal or a metallic alloy as a material for an electrode, the electrode can be formed also by the vacuum deposition method. In the case of using a metal or a metallic alloy as a material forming an electrode, the electrode can be formed, furthermore, by the chemical plating method (see for example, Handbook of Electrochemistry, pp 383-387, Mazuren, 1985). In the case of using an electroconducting polymer, an electrode can be made by forming it into a film by means of anodic oxidation polymerization method onto a substrate, which is previously provided with an electroconducting coating.

As methods for forming said thin films, there are, for example, the vacuum deposition method, the spin-coating method, the casting method, the Langmuir-Blodgett (“LB”) method, the ink jet printing method and the like. Among these methods, the vacuum deposition method, the spin-coating method, the ink jet printing method and the casting method are particularly preferred in view of ease of operation and cost.

In the case of forming the layers by using the spin-coating method, the casting method and ink jet printing method, the coating can be carried out using a solution prepared by dissolving the composition in a concentration of from 0.0001 to 90% by weight in an appropriate organic solvent such as benzene, toluene, xylene, tetrahydrofurane, methyltetra-hydrofurane, N,N-dimethylformamide, acetone, acetonitrile, anisole, dichloromethane, dimethylsulfoxide and mixtures thereof.

Patterning of active matrix OLED (AMOLED) materials for large format, high resolution displays can be done using Laser Induced Thermal Imaging (LITI; Organic Light-Emitting Materials and Devices VII, edited by Zakya H. Kafafi, Paul A. Lane, Proceedings of SPIE Vol. 5519, 12-23).

The organic EL device of the present invention is seen as a future replacement technology for a flat panel display of an on-wall television set, a flat light-emitting device, such as a wall paper, a light source for a copying machine or a printer, a light source for a liquid crystal display or counter, a display signboard and a signal light and perhaps even to replace incandescent and fluorescent lamps. The polymers and compositions of the present invention can be used in the fields of an organic EL device, a photovoltaic device, an electrophotographic photoreceptor, a photoelectric converter, a solar cell, an image sensor, and the like.

Accordingly, the present invention relates also to OLEDs, PLEDs, organic integrated circuits (O—ICs), organic field effect transistors (OFETs), organic thin film transistors (OTFTs), organic solar cells (O—SCs), thermoelectric devices, or organic laser diodes comprising one or more of the polymers according to the present invention.

The present invention is illustrated in more detail on the basis of an especially preferred embodiment below, but should not be limited thereto. In this embodiment the polymer is a homopolymer of the following general formula II

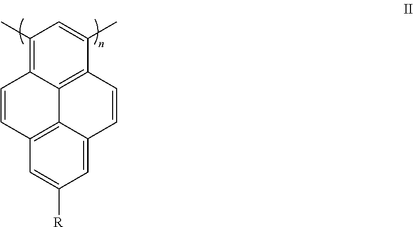

wherein R is an alkyl group, preferably having from 1 to 12 carbon atoms, and n, defining the polymer chain length, is an integer in the range from 20 to 500.

In a more specific embodiment of the invention, R is a tert-butyl group, which is especially advantageous in that the use thereof enables to provide the desired product in a very simple and efficient 3-step-synthesis as outlined in more detail below. However, analogous compounds having other substituents can also be prepared by known methods as indicated below and will exhibit similar favorable characteristics. Additionally, it is also contemplated that one or more of the remaining positions in the pyrene rings which are not substituted in structural formula II may be substituted with a residue which does neither interfere with the method of preparation nor prevents the desired twisted structure of the polymeric molecule described below. Such residues may for example comprise the residues R²-R⁸ as defined in formula I above. Specifically, said residues may comprise a C₁-C₁₂ alkyl group, such as a lower alkyl group, preferably having 1-3 carbon atoms.

In one aspect, the present invention provides effective chemical routes to produce well soluble and processable polypyrenes of formula II having an alkyl substituent in the 7-position of the pyrene ring, in particular, the 7-tert-butyl-1,3-pyrenylene, with a defect-free structure and the highest degree of polymerization reported up to now for polymers made up exclusively from pyrene units.

The addition of alkyl chains to a pyrene ring can be effected by various means, for example from the pyrene-2-boronate starting compound reported in Conventry et al. (Chem. Commun. 2005, 2172-2174), as indicated schematically below:

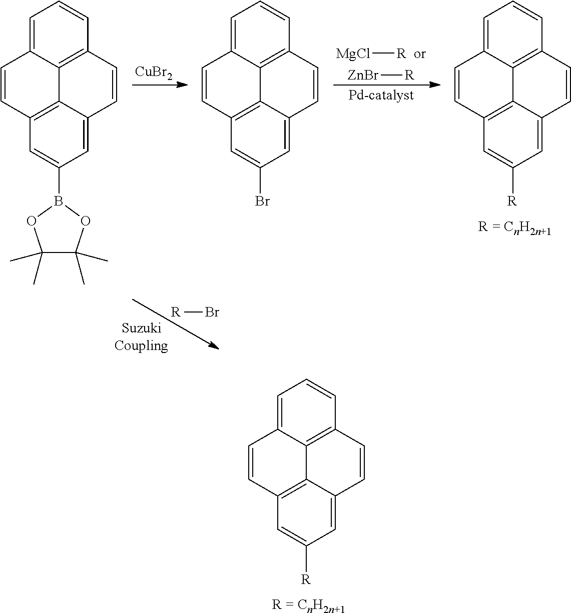

Subsequently, the alkylated pyrene can be further processed and polymerized as indicated in the following general scheme:

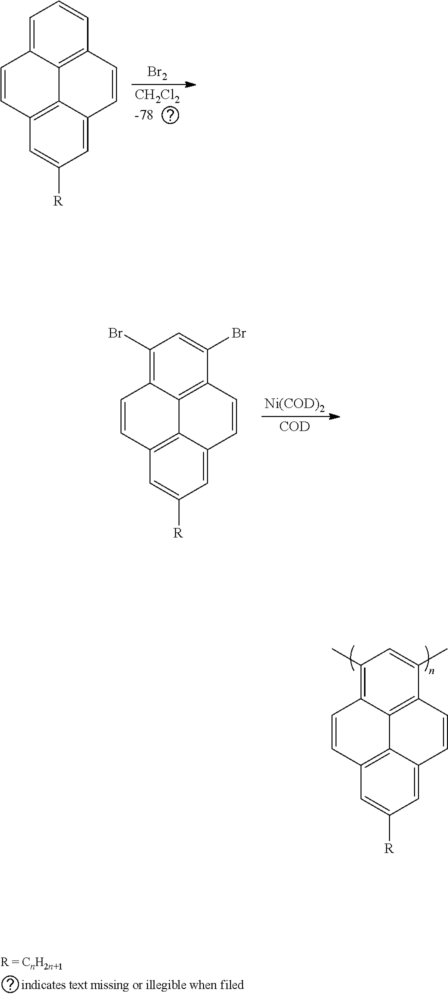

More specifically, poly-7-tert-butyl-1,3-pyrenylene can be produced very effectively in a simple chemical 3-step synthetic route according to the scheme below.

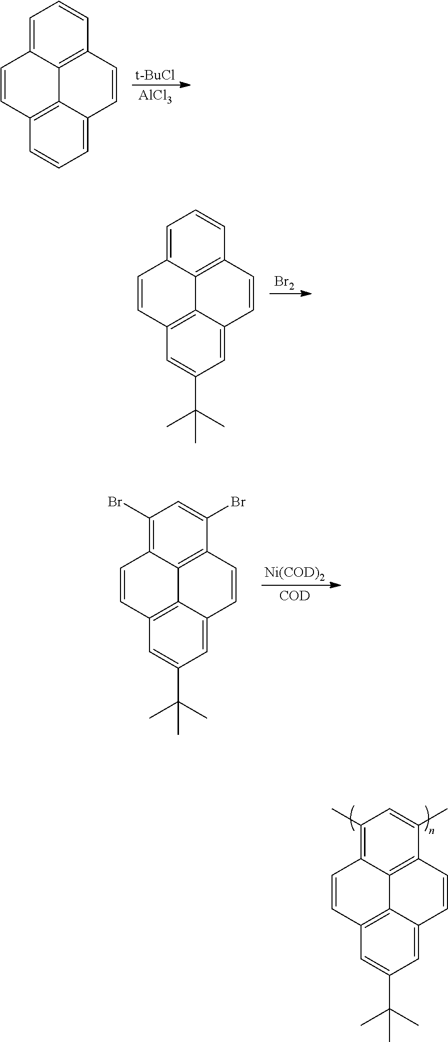

The inventive method for preparing poly-7-tert-butyl-1,3-pyrenylene comprises at least the following steps:
a) mono-tert-butylation pyrene to provide 2-tert-butylpyrene,
b) b) reacting 2-tert-butylpyrene from step a) with bromine or NBS to provide the 1,3-dibromo-7-tert-butylpyrene monomer,
c) polymerization of the monomer in a Yamamoto coupling reaction in the presence of a catalyst.

Preferably, the catalyst in step c) is a Ni(0) catalyst, such as Ni(COD)₂, but other suitable catalysts are available and will be readily recognized by the skilled artisan.

Specifically, in a preferred method for preparing poly-7-tert-butyl-1,3-pyrenylene as used by the present inventors, pyrene was first mono-tert-butylated using a modified protocol of Miura et al., J. Org. Chem. 59, 3294-3300 (1994), to afford 2-tert-butylpyrene, which was then treated with bromine (2 equivalents) in CH₂Cl₂ at −78° C. to provide the 1,3-dibromo-7-tert-butylpyrene in 89% yield. The use of tert-butyl groups was strategic in order to selectively obtain the 1,3-dibromo-7-tert-butylpyrene derivative. The polymerization of 1,3-dibromo-7-tert-butylpyrene was carried out in a Yamamoto coupling reaction with a Ni(0) catalyst, in particular Ni(COD)₂, analogous to Yamamoto, T., in Synlett 4, 425-450 (2003).

As demonstrated by the experimental data detailed below, the resulting poly-7-tert-butyl-1,3-pyrenylene shows a high solid state quantum yield with blue emission, excellent solubility and stability, no aggregation in thin films and excellent electro optical performance in single-layer PLEDs.

### Characterization

After precipitation in a mixture of HCl and methanol (1:1) and subsequent removal of the low-molecular-weight species by Soxhlet extraction with acetone, GPC (gel permeation chromatography) analysis (THF, PPP standards) revealed Mₙ=29800 g/mol, M_{w}=51500 g/mol, and PD=1.7; which corresponds to a molecular structure of approximately 115 repeat units. The resulting polymer exhibits high solubility in common organic solvents (e.g., THF, toluene and chlorinated solvents), enabling the characterization of the polymer by ¹H and ¹³C-NMR spectroscopy and also the investigation of the optical properties in solution. The ¹H-NMR spectra revealed a broad band between 8.7 and 7.0 ppm corresponding to the aromatic protons and a broad signal centered at 1.57 ppm corresponding to the tert-butyl groups, with correct relative signal intensities.

In addition to the high molecular weight, the present polypyrenes demonstrate for the first time the absence of aggregation in such pyrene derivatives which is due to the highly twisted structure of the polymeric chain, leading to important advantages such as good solubility and high fluorescence quantum yield in THF of φ_{f}=0.88 (calculated value using anthracene as reference with excitation at 360 nm) as depicted in FIG. 1. FIG. 1 (upper panel) shows the UV-visible absorption and photoluminescence emission spectra of the polypyrene exhibiting very similar spectra for the thin film and diluted THF solution. The absorption spectra show a π-π* transition at ca. 357 nm with a higher energy absorption band at ca. 280 nm. In contrast, the emission in both solution and in the thin film is characterized by a broad unstructured band with a maximum at 441 nm in solution and a slight bathochromic shift to 454 nm in the solid state. Additionally a minor broadening of the emission spectrum in the solid state is observed.

While the observation of very similar emission and absorption spectra in film and dilute solution already provides good evidence for the absence of aggregation in the excited state, one can exclude its occurrence by performing a classical concentration dependence analysis as depicted in FIG. 1, lower panel, covering 3 orders of magnitude. As clearly observed in this plot, the emission of the dissolved polymer shows no sign of excimer emission. Only above a certain critical concentration is self-absorption of the polymer observed, where filtering out of the lower wavelength edge of the emission spectrum is observed. These findings are direct evidence against any excimer formation in the new polymer. Moreover, since also the emission from aggregates should display a pronounced concentration dependence, which is clearly not observed, we further conclude that no ground state aggregation is taking place. This is supported by the fact that when exciting the polymer in solution at energies close and below the band edge (i.e. at wavelengths higher than 370 nm) we could not find any evidence for an additional emission band or even a shift of the molecular emission.

The absence of excimer and aggregate emission also becomes evident from the calculated molecular structure for a linear 1,3-pentamer model compound (AM1) as shown in the inset of FIG. 2. The modeling clearly shows the twisting between two pyrene rings as a result of strong steric hindrance, giving a dihedral angle of ca. 75°. The twisted structure of the 1,3-pentamer, and likewise of the polymer, drastically reduces self-aggregation due to π-stacking of pyrene units and consequently the self-quenching effect, caused by the formation of aggregates or excimers, leading to solid-state properties which are comparable with those in solution. A comparison of the absorption and emission properties of the polymer with those of a previously synthesized 1,3-trimer (Müllen et al., Angew. Chem. Int. Ed. 47, 10175-10178 (2008) further supports these findings. The 1,3-trimer shows an absorption maximum at 350 nm and an emission maximum at 430 nm in solution compared to 357 nm and 441 nm for the polymer, respectively.

As the morphological stability at high temperature is a critical point for device performance, a thermal characterization of the polypyrene was made using differential scanning calorimetry (DSC) and thermogravimetric analysis (TGA), and the influence of thermal treatment on its optical properties was investigated. The high morphological stability and T_{g} recorded could be attributed to the presence of the rigid pyrene unit in the main chain of the polymer. Furthermore, FIG. 2 shows the emission spectra obtained for a polypyrene film before and after annealing at 150° C. for 24 h under argon atmosphere. It is remarkable that only a slight shift of the emission maximum (4 nm) accompanied by a ca. 8% reduction of the solid-state emission yield was observed after annealing. Similar results were obtained for annealing experiments under ambient conditions. This result indicates that the polymer not only possesses exceedingly thermal stability with respect to morphology but also that the formation of chemical defects upon oxidation at high temperature is sufficiently prevented.

As depicted in FIG. 3, ITO/PEDOT:PSS/polypyrene/CsF/Al OLED structures show bright blue-turquoise electroluminescence with a maximum at 465 nm and a profile very similar to the photoluminescence emission in the solid-state. Luminance values of 300 cd/m² were measured at a bias voltage of 8 V with favourable blue color coordinates of x=0.15 and y=0.32 according to the CIE standard of 1931. The devices show remarkable spectral stability over time with only very minor changes in the spectra as a consequence of a thermal annealing under device operation as depicted in the inset of the graph. The devices display a detectable onset of electroluminescence at approximately 3.5 V and maximum efficiencies of ca 0.3 cd/A. The performance of the presented devices is comparable to devices fabricated without evaporated transport layers from similar poly(para-phenylene)-type based materials with respect to the overall device efficiency and brightness (e.g. J. Jacob et al. J. Am. Chem. Soc., 126, 6987 (2004). However, with respect to overall device stability, the presented polymer shows clear superiority based on its molecular design. In addition, the simple 3-step chemical synthetic route allows for the fabrication of high purity and defect-free polymers. Due to the molecular design, no oxidative degradation processes, as known for non-conjugated polymer segment as “aliphatic CH (photo)oxidation” (M. R. Craig et al., J. Mater. Chem. 13, 286-(2003)) and oxidation of the conjugated chain segments, such as observed for the 9-position in polyfluorene (J. W. List et al., Adv. Mater. 14, 374-(2002)) are detectable in the presented polymers.

In conclusion, the presently claimed compounds, in particular poly-7-tert-butyl-1,3-pyrenylene and related compounds, present the benefit of very stable blue emission, which is a consequence of the 1-3 substitution resulting in a large dihedral angle between pyrene units fully suppressing aggregation and excimer emission. Finally, the simple synthetic route and the high fluorescence quantum yield in thin-films renders these polypyrenes a particularly promising material for PLEDs.

In view of the surprising and advantageous properties of the claimed polypyrenes, further aspects of the present invention relate to various applications thereof.

For example, advantageous applications comprise the use of the claimed compounds as electroluminescent material and/or the use in an electronic device or in a component therefore.

In particular, the polypyrenes may be used as or in the light-emitting active layer of the device. Polypyrenes according to the present invention may be present in the device as the sole light emitting polymer or as a component in a blend further comprising hole and/or electron transporting polymers. Alternatively, the device may comprise distinct layers of polypyrene polymers of the present invention, a hole transporting polymer and/or an electron transporting polymer.

The present polypyrenes may be used in any electronic devices known in the art, e.g. such as disclosed in WO99/48160. Electroluminescent devices are used for example as self-illuminating display elements, such as control lamps, alphanumeric displays, signs and in opto-electronic couplers.

Especially preferred, the inventive polypyrenes are used in polymer light emitting diodes (PLEDs). For this purpose, the polypyrenes of the invention will be typically formed to films and used as emitting layers. The thickness of such layers will be typically in a range of from 0.01 to 0.5 μm.

The films can be prepared by methods well known in the art such as spin-coating, spray-coating, dip-coating and roller-coating. The composition for preparing such a coating will typically contain from 0.1 to 10 weight percent of the polymers. The composition is applied to the appropriate substrate by the desired method and the solvent is allowed to evaporate. Residual solvent may be removed by vacuum and/or by heat-drying. In specific embodiments, some components of the composition may partially cured.

In further related aspects, the present invention therefore also encompasses optical or electronic devices or components therefore and PLEDs, organic integrated circuits (O-Ics), organic field effect transistors (OFETs), organic light-emitting field effect transistors, organic thin film transistors (OTFTs), organic solar cells (O—SCs), thermoelectric devices, electrochromic devices, or organic laser diodes, comprising one or more of the inventive polypyrenes.

### FIGURES

FIG. 1: a) UV-Visible absorption and photoluminescence emission spectra of polymer in THF and in thin film; and b) photoluminescence in toluene at different concentrations ranging from 0.1 mg/l to 1000 mg/l. (Note that the spectrum at highest concentration is affected by self-absorption at wavelengths lower than 520 nm).

FIG. 2: Absolute photoluminescence emission spectra obtained for a polymer film before and after thermal annealing under argon atmosphere at 150° C. for 24 h. The inset shows the molecular model of the linear 1,3-pentamer showing a clearly non-coplanar arrangement of the neighboring pyrene rings as a result of the large steric hindrance.

FIG. 3: Current density (line with squares)/luminance (line with circles) as a function of the bias voltage in an ITO/PEDT:PSS/polypyrene/CsF/Al device. The inset shows the electroluminescence emission spectrum after 1-5 minutes of continuous operation for the same device. The emission spectra have been obtained at bias of 6 V and ca. 400 mA/cm2.

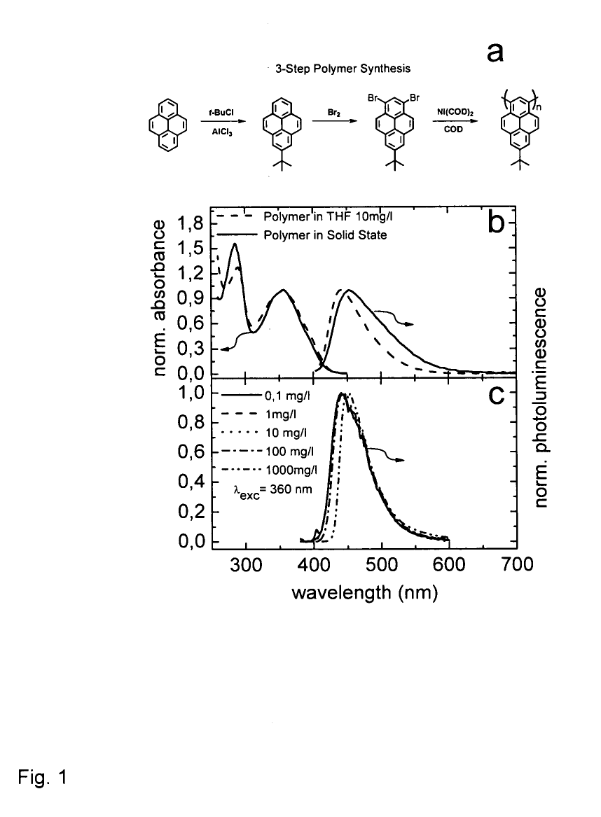

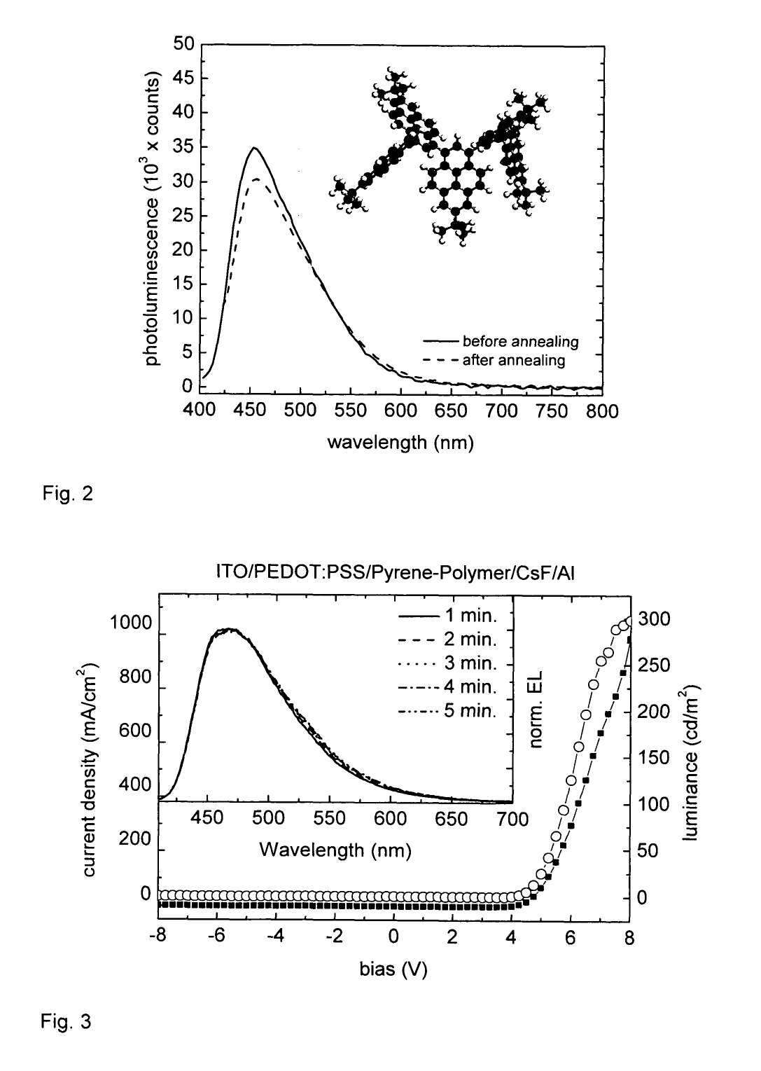

1. A fluorescent polymer comprising repeating unit(s) shown in the following general formula I: 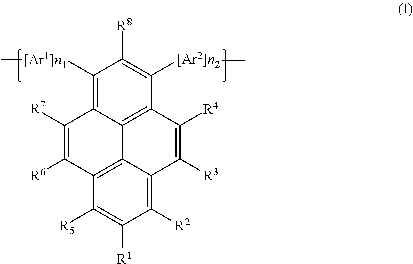
wherein R¹, R², R³, R⁴, R⁵, R⁶, R⁷ and R⁸ are independently of each other hydrogen, halogen, SiR¹⁰⁰R¹⁰¹R¹⁰², or an organic substituent, or
R⁶ and R⁷, R³ and R⁴, and/or any of the substituents R¹, R², R³, R⁴, R⁵, R⁶, R⁷ and/or R⁸, which are adjacent to each other, together form an aromatic, or heteroaromatic ring, or ring system, which can optionally be substituted, n₁ and n₂ are 0, 1, or 2,
R¹⁰⁰, R¹⁰¹ and R¹⁰² are independently of each other C₁-C₁₈ alkyl, substituted or unsubstituted C₆-C₁₈ aryl,
and Ar¹ and Ar² are each independently of each other a substituted or unsubstituted arylene or heteroarylene group.

2. The fluorescent polymer according to claim 1, wherein R¹, R², R³, R⁴, R⁵, R⁶, R⁷ and R⁸ are independently of each other H, halogen, C₁-C₁₈ alkyl, C₁-C₁₈ alkyl which is substituted by E and/or interrupted by D, C₁-C₁₈ perfluoroalkyl, C₆-C₂₄ aryl, C₆-C₂₄ aryl which is substituted by G, C₂-C₂₀ heteroaryl, C₂-C₂₀ heteroaryl which is substituted by G, C₂-C₁₈ alkenyl, C₂-C₁₈ alkynyl, C₁-C₁₈ alkoxy, C₁-C₁₈ alkoxy which is substituted by E and/or interrupted by D, C₇-C₂₅ aralkyl, CN, or —CO—R²⁸;
D is —CO—; —COO—; —S—; —SO—; —SC2—; —O—; —NR²⁵—; —SiR³⁰R³¹—; —POR³²—; —CR²³═CR²⁴—; or —C≡C—; and
E is −OR²⁹; —SR²⁹; —NR²⁵R²⁶; —COR²⁸; —COOR²⁷; —CONR²⁵R²⁶; —CN; or halogen;
G is E, C₁-C₁₈ alkyl, C₁-C₁₈ alkyl which is interrupted by D, C₁-C₁₈ perfluoroalkyl, C₁-C₁₈ alkoxy, or C₁-C₁₈ alkoxy which is substituted by E and/or interrupted by D;
R²³, R²⁴, R²⁵ and R²⁶ are independently of each other H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—;
R²⁷ is H; C₆-C₁₈ aryl; C₆-C₁₈ aryl which is substituted by C₁-C₁₈ alkyl, or C₁-C₁₈ alkoxy; or C₁-C₁₈ alkyl which is interrupted by —O—;
R²⁸ is H; C₆-C₁₈ aryl; C₆-C₁₈ aryl which is substituted by C₁-C₁₈ alkyl, or C₁-C₁₈ alkoxy; C₁-C₁₈ alkyl; or C₁-C₁₈ alkyl which is interrupted by —O—;
R²⁹ is H; C₆-C₁₈ aryl; C₆-C₁₈ aryl, which is substituted by C₁-C₁₈ alkyl, or C₁-C₁₈ alkoxy; C₁-C₁₈ alkyl; or C₁-C₁₈ alkyl which is interrupted by —O—;
R³⁰ and R³¹ are independently of each other C₁-C₁₈ alkyl, C₆-C₁₈ aryl, or C₆-C₁₈ aryl, which is substituted by C₁-C₁₈ alkyl, and
R³² is C₁-C₁₈ alkyl, C₆-C₁₈ aryl, or C₆-C₁₈ aryl, which is substituted by C₁-C₁₈ alkyl.

3. The fluorescent polymer according to claim 1, wherein R², R³, R⁴, R⁵, R⁶, R⁷ and R⁸ are independently of each other H, F, C₁-C₁₈ alkyl, C₁-C₁₈ alkyl which is interrupted by —O—, C₁-C₁₈ alkoxy, or C₁-C₁₈ alkoxy which is interrupted by —O—.

4. The fluorescent polymer according to claim 1, wherein R¹ is C₁-C₁₈ alkyl.

5. The fluorescent polymer according to claim 4, wherein R¹ is branched or unbranched C₁-C₈ alkyl group.

6. The fluorescent polymer according to claim 5, wherein R¹ is a tert-butyl group.

7. The fluorescent polymer according to claim 1, wherein Ar¹ and Ar² are selected from the group consisting of a substituted or unsubstituted benzene, a substituted or unsubstituted naphthalene, a substituted or unsubstituted anthracene, a substituted or unsubstituted diphenylanthracene, a substituted or unsubstituted phenanthrene, a substituted or unsubstituted triphenylene, a substituted or unsubstituted acenaphthene, a substituted or unsubstituted biphenyl, a substituted or unsubstituted fluorene, a substituted or unsubstituted carbazolyl, a substituted or unsubstituted thiophene, substituted or unsubstituted multi-fused thiophenes, a substituted or unsubstituted triazole, a substituted or unsubstituted thiadiazole, a substituted or unsubstituted pyrene, a substituted or unsubstituted triphenylamine, and a perylenediimide or perylenemonoimide or higher rylene homologues thereof.

8. The fluorescent polymer according to claim 1, wherein n₁ and n₂ are 0.

9. The fluorescent polymer according to claim 8, wherein R¹ is a C₁-C₁₂ alkyl group and R², R³, R⁴, R⁵, R⁶, R⁷, and R⁸ are independently of each other hydrogen or C₁-C₁₈ alkyl.

10. The fluorescent polymer according to claim 1, comprising 20 to 1000 repeating units of formula I.

11. The fluorescent polymer according to claim 9 which is a non-aggregating, blue-emitting polypyrene compound having the following general formula II: 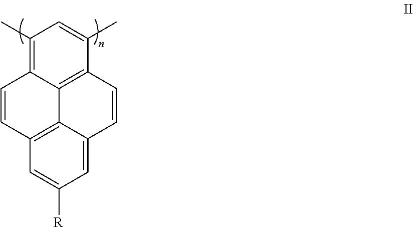
wherein R is an alkyl group having from 1 to 12 carbon atoms, and n is an integer in a range from 20 to 1000.

12. The compound according to claim 11, wherein R is a tert-butyl group.

13. The compound according to claim 11, wherein n is in a range of from 20 to 500.

14. A method for alkylating pyrene rings in the 2- and/or 7-position comprising
a) preparing a pyrene-2-boronate or pyrene-2,7-bis(boronate) compound;
b) reacting the compound of step a) with either: (i) CuBr₂ to obtain a corresponding bromo pyrene derivative which is brominated in the 2- and/or 7-position and subsequently formation in situ of MgCl—R or ZnBr—R in the presence of a palladium catalyst to obtain a corresponding mono- or dialkylated pyrene; or (ii) R—Br in a one-step Suzuki coupling reaction to form the corresponding mono- or dialkylated pyrene.

15. A method for preparing poly-7-alkyl-1,3-pyrenylene comprising the following steps:
a) mono-alkylating pyrene to provide 2-alkylpyrene,
b) reacting 2-alkylpyrene from step a) with a brominating agent to provide the 1,3-dibromo-7-alkylpyrene monomer, and
c) polymerizing the monomer from step b) in a Yamamoto coupling reaction in the presence of a catalyst.

16. The method according to claim 15, wherein the alkyl group is a tert-butyl group.

17. A method of using the compound of claim 1 as an electroluminescent material.

18. A method of using the compound of claim 1 in an electronic device or in a component therefore.

19. A method of using the compound of claim 1 in polymer light emitting diodes (PLEDs).

20. An electronic device or a component therefore, comprising the compound according to claim 1.

21. OLEDS, PLEDs, organic integrated circuits (O-Ics), organic field effect transistors (OFETs), organic light-emitting field effect transistors, organic thin film transistors (OTFTs), organic solar cells (O—SCs), thermoelectric devices, electrochromic devices, or organic laser diodes, comprising one or more of the compounds according to claim 1.
